(19) United States
(12) Patent Application Publication
(10) Pub. No.: US 2021/0113627 A1
(43) Pub. Date: Apr. 22, 2021

(54) ADHERENT STROMAL CELLS DERIVED FROM PLACENTAS OF MULTIPLE DONORS AND USES THEREOF

(71) Applicant: PLURISTEM LTD., Haifa, IL (US)

(72) Inventors: Zami Aberman, Tel-Mond (IL); Ora Burger, Haifa (IL); Shai Meretzki, Haifa (IL)

(21) Appl. No.: 17/119,511

(22) Filed: Dec. 11, 2020

Related U.S. Application Data
(62) Division of application No. 15/655,537, filed on Jul. 20, 2017.
Application No. 15/655,537 is a continuation-in-part of application No. 13/642,725, filed on Oct. 22, 2012, now abandoned.
Application No. 15/655,537 is a continuation-in-part of application No. 12/225,478, filed on Oct. 14, 2009, now abandoned.
(60) Provisional application No. 61/327,330, filed on Apr. 23, 2010.
(60) Provisional application No. 60/847,088, filed on Sep. 26, 2006.
(60) Provisional application No. 60/784,769, filed on Mar. 23, 2006.

(51) Int. Cl. A61K 35/50; A61L 27/38; C12M 3/00; A61P 37/06; C12M 1/12; C12M 1/00; C12N 5/073; C12N 5/0775; C12N 5/00

(52) U.S. Cl. CPC A61K 35/50; A61L 27/3886; C12M 21/08; A61L 27/3895; A61P 37/06; C12N 2500/84; C12M 29/10; C12N 5/0605; A61L 27/3834; C12N 5/0668; C12N 5/0062; C12M 25/14

(57) ABSTRACT

Pharmaceutical compositions comprising adherent stromal cells (ASCs) are provided. The ASCs are obtained from at least two donors. Articles of manufacture comprising the pharmaceutical compositions together with a delivery device for administering the ASCs to a subject are also provided. Also provided are methods of treating various diseases and conditions that are treatable by administering ASCs to a subject in need of treatment.

### CROSS-REFERENCE TO RELATED APPLICATIONS

This application is a divisional of U.S. Ser. No. 15/655,537, filed Jul. 20, 2017, which is a Continuation-in-Part of U.S. Ser. No. 13/642,725, filed Oct. 22, 2012, which is the National Phase of International Application No. PCT/IB2011/001413, filed Apr. 21, 2011, which claims the benefit of priority from Provisional Patent Application 61/327,330, filed Apr. 23, 2010. This application is also a Continuation-in-Part of U.S. Ser. No. 12/225,478, filed Oct. 14, 2009, which is the National Phase of International Application No. PCT/IL2007/000380, filed Mar. 22, 2007, which claims priority to U.S. Provisional Application No. 60/847,088, filed Sep. 26, 2006, and U.S. Provisional Application No. 60/784,769, filed Mar. 23, 2006. The contents of all of the above documents are incorporated by reference as if fully set forth herein.

### BACKGROUND

Pharmaceutical compositions comprising adherent stromal cells (ASCs) are provided. The ASCs are obtained from at least two placentas. Articles of manufacture comprising the pharmaceutical compositions together with a delivery device for administering the ASCs to a subject are also provided. Also provided are methods of treating various diseases and conditions that are treatable by administering ASCs to a subject in need of treatment.

In recent years, considerable activity has focused on the therapeutic potential of mesenchymal stromal cells (MSCs) for various medical applications including tissue repair of damaged organs such as the brain, heart, bone and liver and in support of bone marrow transplantations (BMT). MSCs, a heterogeneous population of cells obtained from, for example, bone marrow, adipose tissue, placenta, or blood, are capable of differentiating into different types of mesenchymal mature cells (e.g. reticular endothelial cells, fibroblasts, adipocytes, osteogenic precursor cells) depending upon influences from various bioactive factors. Accordingly, MSCs have been widely studied in regenerative medicine as the foundation to build new tissues such as bone, cartilage and fat for the repair of injury or replacement of pathologic tissues and as treatment for genetic and acquired diseases [Fibbe and Noort, Ann N Y Acad Sci (2003) 996: 235-44; Horwitz et al., Cytotherapy (2005) 7(5): 393-5; Zimmet and Hare, Basic Res Cardiol (2005) 100(6): 471-81]. Furthermore, the multipotent ability of MSCs, their easy isolation and culture, as well as their high ex vivo expansion potential make them an attractive therapeutic tool [Fibbe and Noort, supra; Minguell et al. Exp Biol Med (Maywood) (2001) 226(6): 507-20].

Placental derived MSCs exhibit many markers common to MSCs isolated from other tissues, for example, CD105, CD73, CD90 and CD29, and the lack of expression of hematopoietic, endothelial and trophoblastic-specific cell markers. Adipogenic, osteogenic, and neurogenic differentiation have been achieved after culturing placental derived MSCs under appropriate conditions [Yen et al., Stem Cells (2005) 23(1): 3-9]. Furthermore, MSCs isolated from placenta and cultured in vitro have been demonstrated to be immune privileged in a similar fashion as bone marrow derived MSCs. Thus, the placenta provides an ethically non-controversial and easily accessible source of MSCs for experimental and clinical applications [Zhang et al., Exp Hematol (2004) 32(7): 657-64].

Methods of making ASCs and using them to treat various conditions are described in International Application No. PCT/IL2008/001185 (published as WO 2009/037690 A1) and International Application No. PCT/IL2009/000527 (published as WO/2009/144720). Those applications describe derivation of ASCs from a single placenta obtained from a single allogeneic donor for transplantation into a subject. As described herein, the inventors have now determined that it is possible to obtain ASCs from multiple placentas, create a mixed ASC population containing ASCs having different HLA types derived from at least two placentas, and then transplant that mixed population into a recipient. This finding, among others, makes it possible to manufacture ASCs by pooling placentas or cells derived from placentas and in that way provides new and useful manufacturing processes and new and useful cell compositions for therapeutic applications, among other things.

### SUMMARY

Compositions and Methods Comprising ASCs from at Least Two Donor Placentas

Provided are methods of treating at least one condition that can be treated by administration of placental-derived adherent stromal cells (ASCs) to a subject in need thereof. In some embodiments the methods include administering to the subject an effective amount of adherent stromal cells (ASCs), wherein the administered ASCs comprise ASCs from at least two donor placentas. In some embodiments, the method comprises administering to a subject an effective amount of ASCs, wherein the ASCs are prepared from at least two donor placentas. In some embodiments the ASCs are obtained by a method comprising culturing placental-derived cells in a three-dimensional (3D) culture. In some embodiments the 3D culturing comprises culturing in a 3D bioreactor. In some embodiments cells in the 3D bioreactor are cultured under perfusion. In some embodiments the 3D bioreactor comprises at least one adherent material selected from a polyester and a polypropylene. In some embodiments the 3D culturing occurs for at least three days. In some embodiments the 3D culture step occurs until at least 10% of the cells are proliferating. In some embodiments the ASCs are positive for at least one marker selected from CD73, CD90, CD29, D7-FIB and CD105. In some embodiments the ASCs from each of the at least two donors are positive for the at least one marker. In some embodiments the ASCs are negative for at least one marker selected from CD3, CD4, CD45, CD80, HLA-DR, CD11b, CD14, CD19, CD34, CD200, KDR, CD31 and CD79. In some embodiments the ASCs from each of the at least two donors are negative for the at least one marker. In some embodiments the ASCs are PLX or PLX-C cells. In some embodiments the ASCs are obtained by a method comprising culturing placental-derived cells in a two-dimensional (2D) culture.

In some embodiments of the methods the at least one condition is selected from stem cell deficiency, heart disease, a neurodegenerative disorder, cancer, stroke, burns, loss of tissue, loss of blood, anemia, an autoimmune disease, ischemia, skeletal muscle regeneration, neuropathic pain, a compromised hematopoietic system, geriatric diseases, and a medical condition requiring connective tissue regeneration and/or repair. In some embodiments the neurodegenerative disorder is selected from multiple sclerosis (MS), Alzheimer's disease, and Parkinson's disease. In some embodiments the ischemia is peripheral arterial disease (PAD). In some embodiments the PAD is critical limb ischemia (CLI). In some embodiments the ischemia comprises ischemia of the central nervous system (CNS). In some embodiments the ischemia is selected from peripheral arterial disease, ischemic vascular disease, ischemic heart disease, ischemic brain disease, ischemic renal disease and ischemic placenta. In some embodiments, the compromised hematopoietic system is caused by radiation or by chemotherapy. In some embodiments the connective tissue comprises at least one of tendon, bone and ligament. In some embodiments the medical condition requiring connective tissue regeneration and repair is selected from bone fracture, bone cancer, burn wound, articular cartilage defect and deep wound. In some embodiments the medical condition requiring connective tissue regeneration and repair is selected from a subchondral-bone cyst, a bone fracture, an osteoporosis, an osteoarthritis, a degenerated bone, a cancer, a cartilage damage, an articular cartilage defect, a degenerative disc disease, an osteogenesis imperfecta (OI), a burn, a burn wound, a deep wound, a delayed wound-healing, an injured tendon and an injured ligament. In some embodiments the autoimmune disease is selected from rheumatoid arthritis, ankylosing spondylitis, inflammatory bowel disease (IBD), MS, diabetes type I, Goodpasture's syndrome, Graves' disease, Hashimoto's disease, Lupus, Myasthenia Gravis, Psoriasis, and Sjorgen's syndrome. In some embodiments the IBD is selected from Crohn's disease and ulcerative colitis.

In some embodiments of the methods the administered ASCs from at least two donors comprise ASCs from at least three, at least four, at least five, at least ten, at least twenty-five, or at least 100 donor placentas. In some embodiments the at least two donors have at least two different HLA genotypes. In some embodiments the at least two different HLA genotypes are genotypes of at least one of the HLA-A, HLA-B, HLA-DR, and HLA-DQ loci. In some embodiments the ASCs from at least two donors are administered to the subject from at least one aliquot. In some embodiments, all of the at least one aliquots comprise ASCs prepared from each of the at least two donors. In some embodiments, one or more of the at least one aliquots comprise ASCs prepared from each of the at least two donors. In some embodiments, one or more of the at least one aliquots comprise ASCs prepared from more than one donor. In some embodiments the aliquot comprising ASCs from each of the at least two donors or the aliquot comprising ASCs from more than one donor is made by a method comprising at least one step selected from mixing placental-derived cells prior to culturing in vitro, mixing placental-derived cells during 2D culturing, mixing placental-derived cells after 2D culturing, mixing placental-derived cells during 3D culturing, and mixing placental-derived cells after 3D culturing. In some embodiments the ASCs from at least two donors are administered to the subject from aliquots each comprising ASCs from only a single donor. In those embodiments in which aliquots comprising ASCs from only a single donor are administered to the patient, ASCs from the aliquot comprising ASCs from one donor may be administered concurrently with, within less than one hour after, within less than 6 hours after, within less than 12 hours after, or within less than 24 hours after ASCs from an aliquot comprising ASCs from any other donor are administered to that patient. In some embodiments the ASCs are administered in one or more treatment courses. In some embodiments, the ASC are administered as one treatment course, two treatment courses, not more than ten treatment courses, ten or more treatment courses, or treatment courses that continue throughout the life of the subject. One treatment course may be separated from another treatment course by 1 day, 2 days, 3 days, 4 days, 5 days, 1 week, 2 weeks, 3 weeks, 1 month, 2 month, 3 months, 4 months, 5 months, 6 months, 1 year, or by 2 or more years. In some embodiments, one treatment course comprises delivering from 1 to 40, from 5 to 40, from 10 to 30, about 5, about 10, about 15, about 20, about 25, about 30, about 35, about 40, or about 45, or about 50 separate injections to the subject. In some embodiments about 30 to about 50 injections of ASCs are administered to the subject for one or two treatment courses. In some embodiments the subject is in need of treatment for critical limb ischemia and the ASCs are administered to the subject in about 30 to about 50 intramuscular injections for one or two treatment courses.

Also provided are pharmaceutical compositions comprising ASCs. In some embodiments the pharmaceutical composition comprises ASCs from at least two donor placentas and a pharmaceutically acceptable carrier. In some embodiments the ASCs are obtained by a method comprising culturing placental-derived cells in a three-dimensional (3D) culture. In some embodiments the 3D culturing comprises culturing in a 3D bioreactor. In some embodiments cells in the 3D bioreactor are cultured under perfusion. In some embodiments the 3D bioreactor comprises at least one adherent material selected from a polyester and a polypropylene. In some embodiments the 3D culturing occurs for at least three days. In some embodiments the 3D culture step occurs until at least 10% of the cells are proliferating. In some embodiments the ASCs are positive for at least one marker selected from CD73, CD90, CD29, D7-FIB and CD105. In some embodiments the ASCs from each of the at least two donors are positive for the at least one marker. In some embodiments the ASCs are negative for at least one marker selected from CD3, CD4, CD45, CD80, HLA-DR, CD11b, CD14, CD19, CD34, CD200, KDR, CD31 and CD79. In some embodiments the ASCs from each of the at least two donors are negative for the at least one marker. In some embodiments the ASCs are PLX or PLX-C cells. In some embodiments the ASCs comprise ASCs from at least three, at least four, at least five, at least ten, at least twenty-five, or at least 100 donors. In some embodiments the at least two donors have at least two different HLA genotypes. In some embodiments the at least two different HLA genotypes are genotypes of at least one of the HLA-A, HLA-B, HLA-DR, and HLA-DQ loci. In some embodiments the ASCs are obtained by a method comprising culturing placental-derived cells in a two-dimensional (2D) culture. In some embodiments the pharmaceutically acceptable carrier is an isotonic solution. In some embodiments the isotonic solution further comprises about 5% human serum albumin. In some embodiments the isotonic solution further comprises about 5% to about 10% dimethyl sulphoxide.

Also provided are articles of manufacture comprising one of the pharmaceutical compositions comprising ASCs and a delivery device for administering the ASCs to a subject. In some embodiments the pharmaceutical composition is packaged within the delivery device. In some embodiments the delivery device is suitable for administering the pharmaceutical composition by intravenous, intramuscular or subcutaneous injection.

### Additional Embodiments of Methods of Cell Expansion, Cells and Conditioned Medium Obtained Thereby, Pharmaceutical Compositions, and Therapeutic Methods

The passages below are intended as a completely separate section of the Summary, unconnected with the previous part of the Summary.

In some embodiments, there is provided a method of cell expansion, the method comprising culturing adherent cells from placenta or adipose tissue under three-dimensional (3D) culturing conditions, which support cell expansion.

According to another aspect, there is provided a method of producing a conditioned medium, the method comprising: culturing adherent cells from a placenta or adipose tissue in 3D culturing conditions which allow cell expansion; and collecting a conditioned medium of the expanded adherent cells, thereby producing the conditioned medium.

According to yet another aspect, there is provided a population of cells generated according to the method as above.

According to still another aspect, there is provided an isolated population of cells comprising adherent cells of placenta or adipose tissue, wherein the adherent cells secrete a higher level of at least one factor selected from the group consisting of SCF, IL-6, and Flt-3 than that secreted by adherent cells of placenta or adipose tissue grown in a 2D culture.

According to an additional aspect, there is provided an isolated population of cells comprising adherent cells of placenta or adipose tissue, wherein the adherent cells express a higher level of at least one protein selected from the group consisting of H2A histone family (H2AF), Aldehyde dehydrogenase X (ALDH X), eukaryotic translation elongation factor 2 (EEEF2), reticulocalbin 3, EF-hand calcium binding domain (RCN2) and calponin 1 basic smooth muscle (CNN1) than that expressed by adherent cells of placenta or adipose tissue grown in a 2D culture.

According to yet an additional aspect, there is provided an isolated population of cells comprising adherent cells of placenta or adipose tissue, wherein the adherent cells express a lower level of expression of at least one protein selected from the group consisting of heterogeneous nuclear ribonucleoprotein H1 (Hnrph1), CD44 antigen isoform 2 precursor, 3 phosphoadenosine 5 phosphosulfate synthase 2 isoform a (Papss2) and ribosomal protein L7a (rpL7a) than that expressed by adherent cells of placenta or adipose tissue grown in a 2D culture.

According to still an additional aspect, there is provided an isolated population of cells comprising adherent cells of placenta or adipose tissue, wherein the adherent cells are characterized by a higher immunosuppressive activity than that of adherent cells of placenta or adipose tissue grown in a 2D culture.

According to further features embodiments described below, the immunosuppressive activity comprises reduction in T cell proliferation.

According to further aspect, there is provided a pharmaceutical composition comprising, as an active ingredient, the population of cells generated according to the method as above.

According to a further aspect, there is provided a pharmaceutical composition comprising, as an active ingredient, the conditioned medium produced according to the method as above.

According to yet a further aspect, there is provided a pharmaceutical composition comprising, as an active ingredient, the isolated population of cells according to above.

According to still a further aspect, there is provided a method of treating a condition which may benefit from stromal cell transplantation in a subject in need thereof, the method comprising administering to the subject a therapeutically effective amount of adherent cells of a tissue selected from the group consisting of placenta and adipose tissue, thereby treating the condition which may benefit from stem cell transplantation in the subject.

According to still a further aspect, there is provided a method of treating a condition which may benefit from stromal cell transplantation in a subject in need thereof, the method comprising administering to the subject a therapeutically effective amount of a conditioned medium of adherent cells derived from a tissue selected from the group consisting of placenta and adipose tissue, thereby treating the condition which may benefit from stem cell transplantation in the subject.

According to still a further aspect, there is provided a method of reducing an immune response in a subject in need thereof, the method comprising administering to the subject a therapeutically effective amount of the described isolated population of cells, so as to reduce the immune response in the subject.

According to still further embodiments, the subject is treated with cell therapy.

According to still further embodiments, the method further comprises administering stem cells.

According to still further embodiments, the stem cells comprise hematopoietic stem cells.

According to still further embodiments, the cells are administered concomitantly with the conditioned medium or adherent cells.

According to still further embodiments, the cells are administered following administration of the conditioned medium or adherent cells.

According to still further embodiments, the adherent cells are obtained from a three dimensional culture.

According to still further embodiments, the adherent cells are obtained from a two dimensional culture.

According to still further embodiments, the condition is selected from the group consisting of stem cell deficiency, heart disease, Parkinson's disease, cancer, Alzheimer's disease, stroke, burns, loss of tissue, loss of blood, anemia, autoimmune disorders, diabetes, arthritis, Multiple Sclerosis, graft vs. host disease (GvHD), neurodegenerative disorders, autoimmune encephalomyelitis (EAE), systemic lupus erythematosus (SLE), rheumatoid arthritis, systemic sclerosis, Sjorgen's syndrome, multiple sclerosis (MS), Myasthenia Gravis (MG), Guillain-Barre Syndrome (OBS), Hashimoto's Thyroiditis (HT), Graves's Disease, Insulin dependent Diabetes Mellitus (IDDM), and Inflammatory Bowel Disease.

According to still further embodiments, the three dimensional culture comprises a 3D bioreactor.

According to still further embodiments, the bioreactor is selected from the group consisting of a plug flow bioreactor, a continuous stirred tank bioreactor and a stationary-bed bioreactor.

According to still further embodiments, the culturing of the cells is effected under a continuous flow of a culture medium.

According to still further embodiments, the 3D culture comprises an adherent material selected from the group consisting of a polyester, a polyalkylene, a polyfluorochloroethylene, a polyvinyl chloride, a polystyrene, a polysulfone, a cellulose acetate, a glass fiber, a ceramic particle, a matrigel, an extracellular matrix component, a collagen, a poly L lactic acid and an inert metal fiber.

According to still further embodiments, the culturing is effected for at least 3 days.

According to still further embodiments, the culturing is effected for at least 3 days.

According to still further embodiments, the culturing is effected until the adherent cells reach at least 60% confluence.

According to still further embodiments, the condition may benefit from the facilitation of hematopoietic stem cell engraftment.

According to still further embodiments, the adherent cells comprise a positive marker expression array selected from the group consisting of CD73, CD90, CD29 and CD105.

According, to still further embodiments, the adherent cells comprise a negative marker expression array selected from the group consisting of CD45, CD80, HLA-DR, CD11b, CD14, CD19, CD34 and CD79.

According to still further embodiments, the adherent cells secrete a higher level of at least one factor selected from the group consisting of SCF, Flt-3 and IL-6 higher than that secreted by adherent cells from placenta or adipose tissue grown in a 2D culture.

According to still further embodiments, the adherent cells express a higher level of at least one protein selected from the group consisting of H2A histone family (H2AF), Aldehyde dehydrogenase X (ALDH X), eukaryotic translation elongation factor 2 (EEEF2), reticulocalbin 3, BF-hand calcium binding domain (RCN2) and calponin 1 basic smooth muscle (CNN1) than that secreted by adherent cells from placenta or adipose tissue grown in a 2D culture.

According to still further embodiments, the adherent cells express a lower level of expression of at least one protein selected from the group consisting of heterogeneous nuclear ribonucleoprotein H1 (Hnrph1), CD44 antigen isoform 2 precursor, 3 phosphoadenosine 5 phosphosulfate synthase 2 isoform a (Papss2) and ribosomal protein L7a (rpL7a) than that secreted by adherent cells from placenta or adipose tissue grown in a 2D culture.

According to still further embodiments, the adherent cells or medium are characterized by a higher immunosuppressive activity than that of adherent cells of placenta or adipose tissue grown in a 2D culture.

According to still further embodiments, the immunosuppressive activity comprises a reduction in T cell proliferation.

According to still further embodiments, the cells comprise cells having a stromal stem cell phenotype.

According to still further embodiments, the stromal stem cell phenotype comprises T cell suppression activity.

According to still further embodiments, the stromal stem cell phenotype comprises hematopoietic stem cell support activity.

According to still further embodiments, the use of the population of cells described above is for manufacture of a medicament identified for transplantation.

The present invention successfully addresses the shortcomings of the presently known configurations by providing novel methods of cell expansion and uses of cells and conditioned medium produced thereby for therapy.

It is to be understood that both the foregoing general description and the following detailed description are exemplary and explanatory only and are not restrictive of the invention, as claimed.

### BRIEF DESCRIPTION OF THE DRAWINGS

FIGS. 1A-G depict the bone-like microenvironment created in the bioreactor system containing 3-D carriers. FIGS. 1A-B are electron micrographs depicting the comparison of natural bone (FIG. 1A) and the structure of the PluriX™ 3D carrier 7 days after seeding Adherent Stromal Cells (3D-ASC), imitating the bone microenvironment (FIG. 1B). FIGS. 1C-F are electron micrographs depicting the PluriX™ 3D matrix seeded with 3D-ASC, produced from bone marrow, 20 days (FIGS. 1C-D, magnified ×150 and 250 respectively) and 40 days (FIGS. 1E-F, magnified ×350 and 500 respectively) after seeding. FIG. 1G is a diagram of the Plurix 3D plug flow bioreactor with separate parts defined by numbers: Culture medium reservoir (1), gas mixture supply (2), filter (3), injection point (4), column in which the 3D carriers are placed (5) flow monitor (6), flow valve (6a), separating container (7), cell growth analyzers (8); peristaltic pump (9), sampling point (10), dissolved 02 measurement electrode (11), pH measurement electrode (12), control system (13), fresh growth media (14), used growth media (15).

FIG. 2 is a graph depicting different production lots of adherent stromal cells (3D-ASC; Lots 5-8) originating from placenta, grown in 3D growth conditions within the bioreactor systems. ASCs (2×10⁶) were seeded in the bioreactor at a density of 10000-15000 cells/a carrier. Following a 12-day culture, 3D-ASCs reached a density of between 150,000-250,000 cells/carrier or 22.5-37.5×10⁶ in a bioreactor containing 150 carriers.

FIGS. 3A-B are bar graphs depicting difference in expression levels of expressed membrane markers in placenta derived 3D-ASC (dark purple) as compared to membrane markers in placenta cells cultured in conventional 2D culture conditions (light purple). Adherent cells were grown for 4-6 weeks in flasks (2D) or for 2-3 weeks in the bioreactor system, on polystyrene carriers (3D). Following harvesting from either flasks or carriers, cells were incubated and bound to a panel of monoclonal antibodies (MAb), which recognize membrane markers characteristic of MSCs (FIG. 3A), or hematopoietic cells (FIG. 3B). Note the significantly higher expression of MSC membrane markers in 2D cultured cells as shown for CD90, CD105, CD73 and CD29 membrane markers, compared to MSC membrane markers expressed in JD-cultured adherent cells, especially CD105 which showed 56% expression in 3D cultured cells vs. 87% in the 2D cultured cells (FIG. 3A). ASCs of both 2D and 3D cultures, did not express any hematopoietic membrane markers (FIG. 3B).

FIGS. 4A-D are bar graphs depicting a comparison of protein levels in ASCs produced from the placenta cultured under 2D and 3D Conditions or conditioned media of same. FIGS. 4A-C depict levels of Flt-3 ligand (FIG. 4A), IL-6 (FIG. 4B) and SCF (FIG. 4C) in pg/ml, normalized for 1×10⁶ cells/ml, as analyzed by ELISA, in the conditioned media of 2D and 3D cultured ASCs. Results represent one of three independent experiments. FIG. 4D shows the expression levels of different cellular proteins, as analyzed by mass spectrometry with iTRAQ reagents labeled protein samples compared therebetween. Protein samples were taken from ASCs grown under 2D (white bars) and 3D (grey bars) conditions. The figure represents one of two replica experiments. Note the difference in expression level of some of the proteins in cells and conditioned media of 2D and 3D culture conditions.

FIG. 5 is a graph depicting percentage of human CD45+ cells detected in bone marrow (BM) of NOD-SCID mice, treated with chemotherapy (25 mg/kg busulfan intraperitoneal injections for two consecutive weeks) 3.5 weeks following transplantation. CD34+ cells (100,000) purified from mononuclear cord blood derived cells, were transplanted alone (5 mice, dataset a) or co-transplanted with 0.5×10⁶ placenta derived adherent cells cultured in 2D conditions (2D-ASC; 2 mice, dataset b), or placenta derived adherent cells cultured in 3D conditions (3D-ASC), in the PluriX™ bioreactor (5 mice, dataset c). BM was then collected from mice femurs and tibias. Human cells in the BM were detected by flow cytometry. The percentage of CD45 expressing human cells was determined by incubating cells with anti-human CD45-FITC. Note the higher percentage of human cells (hCD45+) in the bone marrow of mice co-transplanted with 2D-ASC (dataset b) as well as with 3D-ASC (dataset c) in comparison to the percentage of human cells in the mice treated with HSCs alone (dataset a). The higher engraftment seen in mice treated with 3D-ASC cultured cells in comparison to mice treated with 2D-ASC cultured cells indicates a higher therapeutic advantage unique to 3D cultured ASCs.

FIGS. 6A-B are FACS analyses of human graft CD45+ cells in mice transplanted with CD34+ cells only (FIG. 6A) in comparison to CD34+ cells together with adipose tissue derived ASCs. (FIG. 6B). Note the significantly higher percentage of human hematopoietic population (hCD45+) (FIG. 6A—29%) in a mouse co-transplanted with adipose tissue derived ASC in comparison to a mouse treated with human CD34+ alone (FIG. 6B—12%).

FIG. 7 is a bar graph depicting a mixed lymphocyte reaction conducted between human cord blood mononuclear cells (CB), and equal amounts of irradiated (3000 Rad) cord blood cells (iCB), human peripheral blood derived monocytes (PBMC), 2D cultured (2D) or 3D cultured (3D) placental ASCs, or a combination of PBMC and 2D and 3D cultured placental ASCs (PBMC+2D and PBMC+3D). Size of CB cell population is represented by the ³H-thymidine uptake (measured in CPM) which was measured during the last 18 hours of culturing. Elevation in stimulated CB cell proliferation indicates an immune response of a higher level. Note the lower level of immune response exhibited by cells incubated with adherent cells, and, in particular, the reduction of CB immune response to PBMCs when co-incubated with adherent cells. Three replicates were made of each reaction.

FIG. 8A is a flow chart depicting production of 3D adherent cell from placentas by Celligen™ (designated PLX-C cells). FIG. 8B is a diagram of a Celligen™ bioreactor vessel and ports adapted from The New Brunswick Scientific web site.

FIGS. 9A-C depict expression of fibroblast-typical markers but not expression of endothelial typical markers on PLX-C. FIG. 9A depicts negative expression of the endothelial marker CD31; FIG. 9B depicts negative expression of the endothelial marker KDR; and FIG. 9C depicts positive expression of the human fibroblast marker (D7-FIB). Of note, the histograms shown in the grey/non-bold lines for Isotype IgG1 (FITC) represent the negative control while the histograms shown in the bold lines represent the positively stained cells.

FIGS. 10A-D depict expression of stimulatory and co-stimulatory molecules on PLX-C cells. FIG. 10A depicts PLX-C expression of CD80; FIG. 10B depicts PLX-C expression of CD86; FIG. 10C depicts PLX-C expression of CD40; and FIG. 10D depicts PLX-C expression of HLA-A/B/C. Negative controls were prepared with relevant isotype fluorescence molecules. Of note, histograms shown in the dark grey lines indicate PLX-C marker-expressing population of cells, histograms shown in the bold/black lines indicate bone marrow (BM) marker-expressing population of cells, and histograms shown in the light grey lines indicate mononuclear cell (MNC) marker expressing population of cells.

FIGS. 11A-B depict inhibition of lymphocyte proliferation by PLX-C. FIG. 11A depicts MLR tests performed with 2×10⁵ peripheral blood (PB) derived MNC (donor A) stimulated with equal amount of irradiated (3000 Rad) PB derived MNCs (donor B) followed by addition of increasing amounts of PLX-C cells to the cultures. Three replicates of each group were seeded in 96-well plates. Proliferation rate was measured by [3H]thymidine incorporation; FIG. 11B depicts peripheral blood (PB) derived MNCs stimulated with ConA (1.5 mg/ml). Increasing amounts of PLX-C cells were added to the cultures. Three replicates of each group were seeded in 96-well plates. Proliferation rate was measured by [3H]thymidine incorporation.

FIGS. 12A-C depict PLX-C regulation of pro-inflammatory and anti-inflammatory cytokine secretion following co-culture with peripheral blood cells. FIGS. 12A-B depict secretion of IFNγ (FIG. 12A) and TNFα (FIG. 12B) following co-culture of human derived MNCs (isolated from peripheral blood) stimulated with ConA with PLX-C; FIG. 12C depicts secretion of IFNγ (left bar), TNFα (middle bar) and IL-10 (right bar) following co-culture of human derived MNCs (isolated from peripheral blood) stimulated with LPS with PLX-C. Supernatants were collected and subjected to cytokines analysis using ELISA.

FIG. 13 depicts the average PBMC proliferation rate per bioreactors±SD.

FIG. 14 depicts the average PBMC proliferation per bioreactors±SD.

### DETAILED DESCRIPTION

Example 4 of the Examples section describes methods used to make placenta-derived adherent stromal cells (ASCs). As shown in Example 5, such cells can be administered to a subject either as a population having a common HLA genotype, or as a mixed population of cells having different HLA genotypes. In either case, the data reported in Example 5 demonstrate that allogeneic administration of mixed populations of ASCs does not elicit an immune response in the recipient subject. One ramification of that finding recognized by the inventors is that ASCs from at least two donors may be administered to a subject.

As used herein the phrase “adherent cells” refers to a homogeneous or heterogeneous population of cells which are anchorage dependent in vitro, i.e., which require attachment to a surface or to other cells in order to grow in vitro.

As used herein the term “placenta” refers to any portion of the mammalian female organ which lines the uterine wall and during pregnancy envelopes the fetus, to which it is attached by the umbilical cord. Following birth, the placenta is expelled (and is referred to as a post partum placenta). In some embodiments, “placenta” refers to whole placenta.

Placenta derived adherent cells may be obtained from both fetal (i.e., amnion or inner parts of the placenta, see Example 4) and maternal (i.e., decidua basalis, and decidua parietalis) parts of the placenta. In general, tissue specimens are washed in a physiological buffer [e.g., phosphate-buffered saline (PBS) or Hank's buffer] and single-cell suspensions are made by treating the tissue with a digestive enzyme or a mixture of digestive enzymes (see below) or/and mincing and flushing the tissue parts through a nylon filter or by gentle pipetting with washing medium.

Placenta derived adherent cells can be propagated using two dimensional or three dimensional culturing conditions. Non-limiting examples of such culture conditions are provided in Example 4.

As used herein the phrase “three dimensional culture” refers to a culture in which the cells are exposed to conditions which are compatible with cell growth while allowing the cells to grow in more than one layer. It is well appreciated that the in situ environment of a cell in a living organism (or a tissue) is in a three dimensional architecture. Cells are surrounded by other cells. They are held in a complex network of extra cellular matrix nanoscale fibers that allows the establishment of various local microenvironments. Their extra cellular ligands mediate not only the attachment to the basal membrane but also access to a variety of vascular and lymphatic vessels. Oxygen, hormones and nutrients are ferried to cells and waste products are carried away. The conditions in the three dimensional culture are designed to mimic certain aspects of such an environment as is further exemplified below. It will be appreciated that the conditions of the three-dimensional culture are such that enable expansion of the adherent cells.

As used herein the terms “expanding” and “expansion” refer to substantially differentiation-less maintenance of the cells and ultimately cell growth, i.e., increase of a cell population (e.g., at least 2 fold) without terminal differentiation accompanying such increase.

Examples of adherent materials which may be used to culture cells as described herein include, but are not limited to, a polyester, a polypropylene, a polyalkylene, a polyfluorochloroethylene, a polyvinyl chloride, a polystyrene, a polysulfone, a cellulose acetate, a glass fiber, a ceramic particle, a matrigel, an extra cellular matrix component (e.g., fibronectin, chondronectin, laminin), a collagen, a poly L lactic acid and an inert metal fiber.

Non-limiting examples of base media useful in culturing placental derived cells to derive ASCs include Minimum Essential Medium Eagle, ADC-1, LPM (Bovine Serum Albumin-free), F10 (HAM), F12 (HAM), DCCM1, DCCM2, RPMI 1640, BGJ Medium (with and without Fitton-Jackson Modification), Basal Medium Eagle (BME—with the addition of Earle's salt base), Dulbecco's Modified Eagle Medium (DMEM—without serum), Yamane, IMEM-20, Glasgow Modification Eagle Medium (GMEM), Leibovitz L-15 Medium, McCoy's 5A Medium, Medium M199 (M199E—with Earle's sale base), Medium M199 (M199H—with Hank's salt base), Minimum Essential Medium Eagle (MEM-E—with Earle's salt base), Minimum Essential Medium Eagle (MEM-H—with Hank's salt base) and Minimum Essential Medium Eagle (MEM-NAA with non essential amino acids), among numerous others, including medium 199, CMRL 1415, CMRL 1969, CMRL 1066, NCTC 135, MB 75261, MAB 8713, DM 145, Williams' G, Neuman & Tytell, Higuchi, MCDB 301, MCDB 202, MCDB 501, MCDB 401, MCDB 411, MDBC 153. In some embodiments the medium is DMEM. These and other useful media are available from GIBCO, Grand Island, N.Y., USA and Biological Industries, Bet HaEmek, Israel, among others. A number of these media are summarized in Methods in Enzymology, Volume LVIII, “Cell Culture”, pp. 62 72, edited by William B. Jakoby and Ira H. Pastan, published by Academic Press, Inc.

The medium may be supplemented such as with serum such as fetal serum of bovine or other species, and optionally or alternatively, growth factors, vitamins (e.g. ascorbic acid), cytokines, salts (e.g. B-glycerophosphate), steroids (e.g. dexamethasone) and hormones e.g., growth hormone, erythropoietin, thrombopoietin, interleukin 3, interleukin 6, interleukin 7, macrophage colony stimulating factor, c-kit ligand/stem cell factor, osteoprotegerin ligand, insulin, insulin like growth factors, epidermal growth factor, fibroblast growth factor, nerve growth factor, cilary neurotrophic factor, platelet derived growth factor, and bone morphogenetic protein at concentrations of between picogram/ml to milligram/ml levels.

The skilled artisan will appreciate that additional components may be added to the culture medium. Such components may be antibiotics, antimycotics, albumin, amino acids, and other components known to the art for the culture of cells. Additionally, components may be added to enhance the differentiation process when desirable.

As mentioned, once adherent cells are at hand they may be passaged to two dimensional or three dimensional settings (see Example 4). It will be appreciated though, that alternative embodiments are also possible in which the cells are transferred to a 3D-configured matrix immediately after isolation or alternatively, may be passaged to three dimensional settings following two dimensional conditions.

Thus, the adherent material is configured for 3D culturing thereby providing a growth matrix that substantially increases the available attachment surface for the adherence of the cells so as to mimic the infrastructure of the tissue (e.g., placenta).

Examples of 3D bioreactors include, but are not limited to, a plug flow bioreactor, a continuous stirred tank bioreactor, a stationary-bed bioreactor, a CelliGen Plus® bioreactor system (New Brunswick Scientific (NBS) or a BIOFLO 310 bioreactor system (New Brunswick Scientific (NBS).

As shown Example 4, the Celligen bioreactor is capable of 3D expansion of adherent cells under controlled conditions (e.g., pH, temperature and oxygen levels) and with constant cell growth medium perfusion. Furthermore, the cell cultures can be directly monitored for concentration levels of glucose, lactate, glutamine, glutamate and ammonium. The glucose consumption rate and the lactate formation rate of the adherent cells can be used to measure cell growth rate and to determine the harvest time.

Other 3D bioreactors that can be used include, but are not limited to, a continuous stirred tank bioreactor [where a culture medium is continuously fed into the bioreactor and a product is continuously drawn out to maintain a time-constant steady state within the reactor], a stirred tank bioreactor with a fibrous bed basket [available, for example, from New Brunswick Scientific Co., Edison, N.J.], a stationary-bed bioreactor, an air-lift bioreactor [where air is typically fed into the bottom of a central draught tube flowing up while forming bubbles, and disengaging exhaust gas at the top of the column], a cell seeding perfusion bioreactor with Polyactive foams [as described in Wendt, D. et al., Biotechnol Bioeng 84: 205-214, (2003)], and tubular poly-L-lactic acid (PLLA) porous scaffolds in a Radial-flow perfusion bioreactor [as described in Kitagawa et al., Biotechnology and Bioengineering 93(5): 947-954 (2006)]. Other bioreactors which can be used are described in U.S. Pat. Nos. 6,277,151, 6,197,575, 6,139,578, 6,132,463, 5,902,741 and 5,629,186.

Cell seeding is preferably effected at a concentration of 100,000-1,500,000 cells/ml at seeding. In an exemplary embodiment a total of 150±30×10⁶ cells are seeded, 3-5×10⁶ cell/g carrier are seeded, or 0.015-0.1×10⁶ cell/ml are seeded.

In some embodiments the ASCs are positive for at least one marker selected from CD73, CD90, CD29, D7-FIB and CD105. A population is positive for a marker if the population contains a proportion of cells positive for the marker such that expression of the marker above a threshold level can be detected in the population as a whole. Threshold levels can be determined, for example, by comparison to a known negative population of cells, by omission of a reagent used in the detection protocol, or by substitution of a non-detecting reagent, such as an isotype control, in the detection protocol. In some embodiments expression is measured on a cell by cell basis, such as using a FACS analysis, while in others it is measured on an entire sample of the population at once, such as using a Western Blot. In some embodiments positive expression of the marker in the population is defined as detectable expression by at least 5%, at least 10%, at least 20%, or at least 50% or more of the cells in the population.

In some embodiments the ASCs are negative for at least one marker selected from CD3, CD4, CD45, CD80, HLA-DR, CD11b, CD14, CD19, CD34, CD200 and CD79. A population is negative for a marker if the population contains so few cells positive for the marker that expression of the marker above a threshold level can not be detected in the population as a whole. Threshold levels can be determined, for example, by comparison to a known negative population of cells, by omission of a reagent used in the detection protocol, or by substitution of a non-detecting reagent, such as an isotype control, in the detection protocol. In some embodiments expression is measured on a cell by cell basis, such as using a FACS analysis, while in others it is measured on an entire sample of the population at once, such as using a Western Blot. In some embodiments negative expression of the marker in the population is defined as detectable expression by less than 50%, less than 20%, less than 10%, and less than 5% of the cells in the population.

A pharmaceutical composition comprising ACSs from at least two donor placentas can be formed by mixing placental-derived cells at any point following harvesting of a placenta. By way of non-limiting example, the pharmaceutical composition can be made by a method comprising at least one step selected from mixing placental-derived cells prior to culturing in vitro, mixing placental-derived cells during 2D culturing, mixing placental-derived cells after 2D culturing, mixing placental-derived cells during 3D culturing, and mixing placental-derived cells after 3D culturing. The compositions comprising the ASCs may be subdivided and stored as aliquots comprising at least one effective amount of the ASCs. The aliquots may be prepared in tubes, bags, or any other container suitable for preserving the at least one effective amount of ASCs for use in the methods described.

In some embodiments, the ASCs are capable of suppressing immune reaction in a subject. As used herein the phrase “suppressing immune reaction in a subject” refers to decreasing or inhibiting the immune reaction occurring in a subject in response to an antigen (e.g., a foreign cell or a portion thereof). The immune response which can be suppressed by the adherent cells include the humoral immune responses, and cellular immune responses, which involve specific recognition of pathogen antigens via antibodies and T-lymphocytes (proliferation of T cells), respectively.

As used herein the term “treating” refers to inhibiting or arresting the development of a disease or condition (e.g., ischemia) and/or causing the reduction, remission, or regression of the disease or condition. In some embodiments the inhibition or arrest is accompanied by the reduction, remission, or regression or at least one symptom of the disease or condition. Those of skill in the art will understand that various methodologies and assays can be used to assess the development of a disease or condition, and similarly, various methodologies and assays may be used to assess the reduction, remission or regression of a disease or condition.

The phrase “connective tissue” refers to a supporting framework tissue comprising strands of collagen, elastic fibers (e.g., between and around muscle and blood vessels) and simple cells. Examples of connective tissues include, but are not limited to dense connective tissue (e.g., ligament, tendon, periodontal ligament), areolar connective tissue (e.g., with proteinaceous fibers such as collagen and elastin), reticular connective tissue, adipose tissue, blood, bone, cartilage, skin, intervertebral disc, dental pulp, dentin, gingival, extracellular matrix (ECM)-forming cells, loose connective tissue and smooth muscle cells.

The term “ischemia” as used herein refers to any pathology (disease, condition, syndrome or disorder) characterized by or associated with insufficient angiogenesis. Examples include, but are not limited to, a peripheral arterial disease (PAD) such as limb ischemia and critical limb ischemia (CLI), ischemic heart disease, ischemic brain disease (e.g. stroke), delayed wound-healing, delayed ulcer healing, reproduction associated disorders, arteriosclerosis, ischemic vascular disease, ischemic heart disease, myocardial ischemia, coronary artery disease (CAD), atherosclerotic cardiovascular disease, left main coronary artery disease, arterial occlusive disease, peripheral ischemia, peripheral vascular disease, vascular disease of the kidney, peripheral arterial disease, limb ischemia, lower extremity ischemia, cerebral ischemia, cerebro vascular disease, retinopathy, retinal repair, remodeling disorder, von Hippel-Lindau syndrome, hereditary hemorrhagic telengiectasiaischemic vascular disease, Buerger's disease, ischemic renal disease and ischemic placenta.

As used herein the phrase “medical condition requiring connective tissue regeneration and/or repair” refers to any pathology characterized by connective tissue damage (i.e., non-functioning tissue, cancerous or pre-cancerous tissue, broken tissue, fractured tissue, fibrotic tissue, or ischemic tissue) or loss (e.g., following a trauma, an infectious disease, a genetic disease, and the like). Non-limiting examples of such pathologies include, bone fracture, bone cancer (e.g., osteosarcoma, bone cancer metastasis), burn wound, articular cartilage defect and deep wound.

Since non-autologous cells may induce an immune reaction when administered to the body several approaches have been developed to reduce the likelihood of rejection of non-autologous cells. These include, for example, either suppressing the recipient immune system or encapsulating the non-autologous cells in immunoisolating, semipermeable membranes before transplantation.

Encapsulation techniques are generally classified as microencapsulation, involving small spherical vehicles and macroencapsulation, involving larger flat-sheet and hollow-fiber membranes (Uludag, H. et al. Technology of mammalian cell encapsulation. Adv Drug Deliv Rev. 2000; 42: 29-64).

Methods of preparing microcapsules are known in the arts and include for example those disclosed by Lu M Z, et al., Cell encapsulation with alginate and alpha-phenoxycinnamylidene-acetylated poly(allylamine). Biotechnol Bioeng. 2000, 70: 479-83, Chang T M and Prakash S. Procedures for microencapsulation of enzymes, cells and genetically engineered microorganisms. Mol. Biotechnol. 2001, 17: 249-60, and Lu M Z, et al., A novel cell encapsulation method using photosensitive poly(allylamine alpha-cyanocinnamylideneacetate). J. Microencapsul. 2000, 17: 245-51.

For example, microcapsules are prepared by complexing modified collagen with a ter-polymer shell of 2-hydroxyethyl methylacrylate (HEMA), methacrylic acid (MAA) and methyl methacrylate (MMA), resulting in a capsule thickness of 2-5 μm. Such microcapsules can be further encapsulated with additional 2-5 μm ter-polymer shells in order to impart a negatively charged smooth surface and to minimize plasma protein absorption (Chia, S. M. et al. Multi-layered microcapsules for cell encapsulation Biomaterials. 2002 23: 849-56).

Other microcapsules are based on alginate, a marine polysaccharide (Sambanis, A. Encapsulated islets in diabetes treatment. Diabetes Technol. Ther. 2003, 5: 665-8) or its derivatives. For example, microcapsules can be prepared by the polyelectrolyte complexation between the polyanions sodium alginate and sodium cellulose sulphate with the polycation poly(methylene-co-guanidine) hydrochloride in the presence of calcium chloride.

It will be appreciated that cell encapsulation is improved when smaller capsules are used. Thus, the quality control, mechanical stability, diffusion properties, and in vitro activities of encapsulated cells improved when the capsule size was reduced from 1 mm to 400 μm (Canaple L. et al., Improving cell encapsulation through size control. J Biomater Sci Polym Ed. 2002; 13:783-96). Moreover, nanoporous biocapsules with well-controlled pore size as small as 7 nm, tailored surface chemistries and precise microarchitectures were found to successfully immunoisolate microenvironments for cells (Williams D. Small is beautiful: microparticle and nanoparticle technology in medical devices. Med Device Technol. 1999, 10: 6-9; Desai, T. A. Microfabrication technology for pancreatic cell encapsulation. Expert Opin Biol Ther. 2002, 2: 633-46).

Examples of immunosuppressive agents include, but are not limited to, methotrexate, cyclophosphamide, cyclosporine, cyclosporin A, chloroquine, hydroxychloroquine, sulfasalazine (sulphasalazopyrine), gold salts, D-penicillamine, leflunomide, azathioprine, anakinra, infliximab (REMICADE), etanercept, TNFαblockers, a biological agent that targets an inflammatory cytokine, and Non-Steroidal Anti-Inflammatory Drug (NSAIDs). Examples of NSAIDs include, but are not limited to acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors and tramadol. Conditions and Diseases that can be Treated with ASCs

Peripheral arterial disease (PAD) is a chronic disease that progressively restricts blood flow in the limbs that can lead to serious medical complications. This disease is often associated with other clinical conditions, including hypertension, cardiovascular disease, hyperlipidemia, diabetes, obesity and stroke. Critical Limb Ischemia (CLI) is used to describe patients with chronic ischemia induced pain, ulcers, tissue loss or gangrene in the limb. CLI represents the end stage of PAD patients who need comprehensive treatment by a vascular surgery or vascular specialist. In contrast to coronary and cerebral artery disease, peripheral arterial disease (PAD) remains an under-appreciated condition that despite being serious and extremely prevalent is rarely diagnosed and even less frequently treated. Consequently, CLI often leads to amputation or death and mortality rates in PAD patients exceed that of patients with myocardial infarction and stroke.

In attempts to treat ischemic conditions, various adult stem cells have been used. Thus, co-culturing of adipose tissue derived stromal cells (ADSC) and endothelial cells (EC) resulted in a significant increase in EC viability, migration and tube formation mainly through secretion of VEGF and HGF. Four weeks after transplantation of the stromal cells into the ischemic mouse hind limb the angiogenic scores were improved [Nakagami et al., J Atheroscler Thromb (2006) 13(2): 77-81]. Moon et al. [Cell Physiol Biochem. (2006) 17: 279-90] have tested the ability of ADSC to treat limb ischemia in immunodeficient mice and demonstrated a significant increase in the laser Doppler perfusion index in ADSC-transplanted group.

In addition, when umbilical cord blood (UCB)-derived mesenchymal stem cells were transplanted into four men with Buerger's disease who had already received medical treatment and surgical therapies, ischemic rest pain, suddenly disappeared from their affected extremities [Kim et al., Stem Cells (2006) 24(6): 1620-6]. Moreover, transplantation of human mesenchymal stem cells isolated from fetal membranes of term placenta (FMhMSC) into infarcted rat hearts was associated with increased capillary density, normalization of left ventricular function, and significant decrease in scar tissue, which was enhanced when the stem cells were preconditioned with a mixed ester of hyaluronan with butyric and retinoic acid [Ventura et al., (2007) J. Biol. Chem., 282: 14243-52].

Stroke is one of the leading causes of death around the world. Although there has been a constant reduction in stroke mortality in developed countries, probably due to improved control of stroke risk factors (especially high blood pressure, diabetes and cigarette smoking), stroke still leads to permanent damage (e.g. tissue damage, neurological damage).

New treatment regimens for stroke include stem cell therapy. Transplantation of stem cells or progenitors into the injured site, either locally or via intravenous routes, to replace nonfunctional cells, enhance proliferation and/or differentiation of endogenous stem or progenitor cells and supply necessary immune modulators has been contemplated and stand as the major cell-based strategy. Potential sources of stem/progenitor cells for stroke include fetal neural stem cells, embryonic stem cells, neuroteratocarcinoma cells, umbilical cord blood-derived non-hematopoietic stem cells, bone marrow-derived stem cells and placental-derived mesenchymal stem cells [Andres et al., Neurosurg Focus (2008) 24(3-4): E16].

In a recent study, Koh et. al. [Koh et al., Brain Res. (2008)] examined the neuroprotective effects and mechanisms of implanted human umbilical cord-derived mesenchymal stem cells (hUC-MSCs) in an ischemic stroke rat model. Twenty days after the induction of in-vitro neuronal differentiation, hUC-MSCs displayed morphological features of neurons and expressed neuronal cell markers and neuronal factors (e.g. glial cell line-derived neurotrophic factor, brain-derived neurotrophic factor). Furthermore, in-vivo implantation of the hUC-MSCs into the damaged hemisphere of immunosuppressed ischemic stroke rats improved neurobehavioral function and reduced infarct volume relative to control rats. Three weeks after implantation, hUC-MSCs were present in the damaged hemisphere and expressed neuron-specific markers, yet these cells did not become functionally active neuronal cells.

Various conditions and pathologies require connective tissue (e.g., bone, tendon and ligament) regeneration and/or repair. These include, for example, bone fractures, burns, burn wound, deep wound, degenerated bone, various cancers associated with connective tissue loss (e.g., bone cancer, osteosarcoma, bone metastases), and articular cartilage defect.

The use of autologous BM-MSCs to enhance bone healing has been described for veterinary and human orthopedic applications and include percutaneous injection of bone marrow for ligament healing (Carstanjen et al., 2006), treatment of bone defects by autografts or allografts of bone marrow in orthopedic clinic (Horwitz et al., 1999, Horwitz et al., 2002), regeneration of critical-sized bone defect in dogs using allogeneic [Arinzeh T L, et al., J Bone Joint Surg Am. 2003, 85-A (10):1927-35] or autologous [Bruder S P, et al., J Bone Joint Surg Am. 1998 July; 80 (7):985-96] bone marrow-MSCs loaded onto ceramic cylinder consisting of hydroxyapatite-tricalcium phosphate, or in rabbit using allogeneic peripheral blood derived MSCs (Chao et al., 2006), and extensive bone formation using MSCs implantation in baboon (Livingston et al, 2003).

Within the equine orthopedic field, mesenchymal stem cells of BM and adipose sources have been used experimentally for surgical treatment of subchondral-bone cysts, bone fracture repair [Kraus and Kicker-Head, Vet Surg (2006) 35(3): 232-42] and cartilage repair [Brehm et al., Osteoarthritis Cartilage (2006) 14(12): 1214-26; Wilke et al., J Orthop Res (2007) 25(7): 913-25] and clinically in the treatment of overstrain induced injuries of tendons in horses. Furthermore, different therapeutic approaches have been used to promote suspensory ligament healing in horses (Herthel, 2001). Herthel (2001) have demonstrated a novel biological approach to facilitate suspensory ligament healing that involves the intra lesional injection of autologous stem cells and associated bone marrow components to stimulate natural ligament regeneration.

Rabbit models for injured tendons showed that MSC-treated tissues were stronger and stiffer than natural repaired tissues (Gordon et al., 2005). In addition, seeding of cultured MSCs into a tendon gap resulted in significantly improved repair biomechanics (Young et al., 1998, Osiris Therapeutics, www.osiris.com). Osiris Chondrogen (adult Mesenchymal Stem Cells) is being tested in patients in order to evaluate safety and efficacy. In MSC treated animals, surgically removed meniscal tissue was regenerated, the cartilage surface was protected, and lessened joint damage was observed in comparison to control animals. These benefits persisted in animal models at least through one year (Osiris Therapeutics, www.osiris.com).

Inflammatory bowel disease (IBD), a group of inflammatory conditions of the large intestine and small intestine, includes Crohn's disease and ulcerative colitis and is a chronic, relapsing, and remitting condition of an unknown origin. Crohn's disease (also known as granulomatous colitis and regional enteritis), an autoimmune disease caused by the immune system's attacking the gastrointestinal tract and producing inflammation in the gastrointestinal tract, is an inflammatory disease that may affect any part of the gastrointestinal tract from mouth to anus, causing a wide variety of symptoms. It primarily causes abdominal pain, diarrhea, vomiting and weight loss, but may also cause complications outside of the gastrointestinal tract such as skin rashes, arthritis and inflammation of the eye. There is currently no known drug or surgical cure for Crohn's disease and treatment options are restricted to controlling symptoms, maintaining remission and preventing relapse (using, e.g., 5-aminosalicylic acid (5-ASA) formulations, corticosteroids such as prednisone and hydrocortisone, immunomodulators such as azathioprine and mercaptopurine, and biologic anti-tumor necrosis factor alpha agents). Ulcerative colitis, a form of colitis, is a disease of the intestine, specifically the large intestine or colon that includes characteristic ulcers, or open sores, in the colon. The main symptom of active disease is usually constant diarrhea mixed with blood. Current treatment of ulcerative colitis is similar to Crohn's disease. Colectomy (partial or total removal of the large bowel through surgery) is occasionally necessary. The use of ASCs in the treatment of inflammatory diseases of the colon is described in WO2009/144720, published 3 Dec. 2009, which is incorporated herein by reference.

### Additional Embodiments of Methods of Cell Expansion, Cells and Conditioned Medium Obtained Thereby, Pharmaceutical Compositions, and Therapeutic Methods

The passages below are intended as a completely separate section of the Detailed Description, unconnected with the previous part of the Detailed Description.

Certain embodiments related to novel methods of cell expansion and uses of cells and conditioned medium produced thereby, for stem cell related therapy, stem cell engraftment and HSC support.

The present inventors have uncovered that adherent cells from placenta or adipose tissue can be efficiently propagated in 3D culturing conditions. Surprisingly, the present inventors uncovered that such cells comprise functional properties which are similar to those of MSCs and therefore these cells and the conditioned medium produced there from, can be used for therapeutic purposes such as transplantation, tissue regeneration and in vivo HSC support.

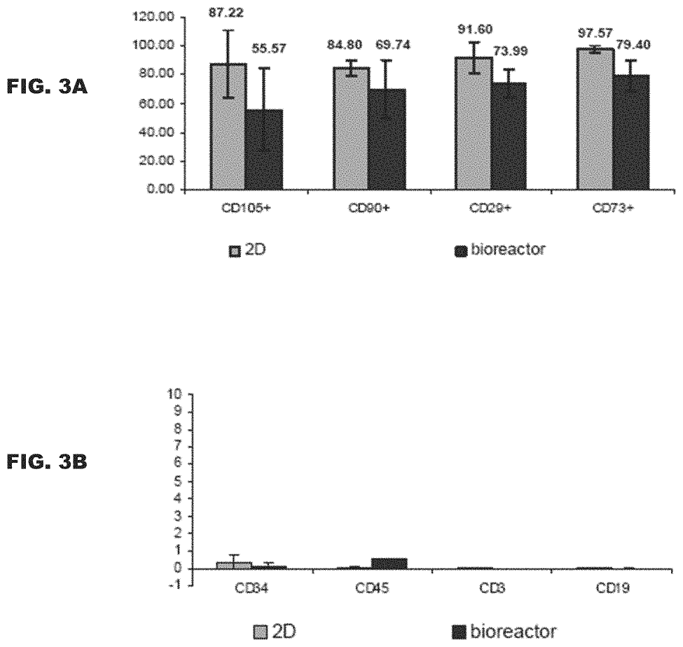

As is illustrated herein below and in Examples 1-3 of the Examples section which follows, the present inventors were able to expand adipose and placenta-derived adherent cells which comprise stromal stem cells properties in 3D settings. Cells expanded accordingly were found viable, following cryo-preservation, as evidenced by adherence and re-population assays (see Example 1). Flow cytometry analysis of placenta-derived adherent cells uncovered a distinct marker expression pattern and (see FIGS. 3A-B). Most importantly, adipose and placenta derived adherent cells propagated on 2D or 3D settings were able to support HSC engraftment (see Example 2), substantiating the use of the described cells, as stromal stem cells, in the clinic.

Thus, according to one embodiment, there is provided a method of cell expansion.

The method comprising culturing adherent cells from placenta or adipose tissue under three-dimensional (3D) culturing conditions which support cell expansion.

As used herein the terms “expanding” and “expansion” refer to substantially differentiationless maintenance of the cells and ultimately cell growth, i.e., increase of a cell population (e.g., at least 2 fold) without differentiation accompanying such increase.

As used herein the terms “maintaining” and “maintenance” refer to substantially differentiationless cell renewal, i.e., substantially stationary cell population without differentiation accompanying such stationarity.

As used herein the phrase “adherent cells” refers to a homogeneous or heterogeneous population of cells which are anchorage dependent, i.e., require attachment to a surface in order to grow in vitro.

As used herein the phrase “adipose tissue” refers to a connective tissue which comprises fat cells (adipocytes).

As used herein the term “placenta tissue” refers to any portion of the mammalian female organ which lines the uterine wall and during pregnancy envelopes the fetus, to which it is attached by the umbilical cord. Following birth, the placenta is expelled (and is referred to as a post partum placenta).

As used herein the phrase “three dimensional culturing conditions” refers to disposing the cells to conditions which are compatible with cell growth while allowing the cells to grow in more than one layer. It is well appreciated that the in situ environment of a cell in a living organism (or a tissue) as a three dimensional architecture. Cells are surrounded by other cells. They are held in a complex network of extra cellular matrix nanoscale fibers that allows the establishment of various local microenvironments. Their extra cellular ligands mediate not only the attachment to the basal membrane but also access to a variety of vascular and lymphatic vessels. Oxygen, hormones and nutrients are ferried to cells and waste products are carried away. The described 3D culturing conditions are designed to mimic such as environment as is further exemplified below.

Thus, adherent cells of this embodiment are retrieved from an adipose or placental tissue.

Placental cells may be obtained from a full-term or pre-term placenta. In certain embodiments, the placenta is perfused for a period of time sufficient to remove residual cells. The term “perfuse” or “perfusion” used herein refers to the act of pouring or passaging a fluid over or through an organ or tissue. The placental tissue may be from any mammal; most preferably the placental tissue is human. A convenient source of placental tissue is from a post partum placenta (e.g., 1-6 hours), however, the source of placental tissue or cells or the method of isolation of placental tissue is not critical to the invention.

Placenta derived adherent cells may be obtained from both fetal (i.e., amnion or inner parts of the placenta, see Example 1) and maternal (i.e., decidua basalis, and decidua parietalis) parts of the placenta. Tissue specimens are washed in a physiological buffer [e.g., phosphate-buffered saline (PBS) or Hank's buffer). Single-cell suspensions are made by treating the tissue with a digestive enzyme (see below) or/and mincing and flushing the tissue parts through a nylon filter or by gentle pipetting (Falcon, Becton, Dickinson, San Jose, Calif.) with washing medium.

Adipose tissue derived adherent cells may be isolated by a variety of methods known to those skilled in the art. For example, such methods are described in U.S. Pat. No. 6,153,432. The adipose tissue may be derived from omental/visceral, mammary, gonadal, or other adipose tissue sites. A preferred source of adipose tissue is omental adipose. In humans, the adipose is typically isolated by liposuction.

Isolated adherent cells from adipose tissue may be derived by treating the tissue with a digestive enzyme such as collagenase, trypsin and/or dispase; and/or effective concentrations of hyaluronidase or DNAse; and ethylenediaminetetra-acetic acid (EDTA); at temperatures between 25-50° C., for periods of between 10 minutes to 3 hours. The cells may then be passed through a nylon or cheesecloth mesh filter of between 20 microns to 800 microns. The cells are then subjected to differential centrifugation directly in media or over a Ficoll or Percoll or other particulate gradient. Cells are centrifuged at speeds of between 100 to 3000×g for periods of between 1 minutes to 1 hour at temperatures of between 4-50° C. (see U.S. Pat. No. 7,078,230).

In addition to placenta or adipose tissue derived adherent cells, also envisaged is the use of adherent cells from other cell sources which are characterized by stromal stem cell phenotype (as will be further described herein below). Tissue sources from which adherent cells can be retrieved include, but are not limited to, cord blood, hair follicles [e.g. as described in Us Pat. App. 20060172304], testicles [e.g., as described in Guan K., et al., Nature. 2006 Apr. 27; 440(7088):1199-203], human olfactory mucosa [e.g., as described in Marshall, C T., et al., Histol Histopathol. 2006 June; 21(6):633-43], embryonic yolk sac [e.g., as described in Geijsen N, Nature. 2004 Jan. 8; 427(6970):148-54] and amniotic fluid [Pietemella et al. (2004) Stern Cells 22:1338-1345], all of which are known to include mesenchymal stem cells. Adherent cells from these tissue sources can be isolated by culturing the cells on an adherent surface, thus isolating adherent cells from other cells in the initial population.

Regardless of the origin (e.g., placenta or adipose tissue), cell retrieval is preferably effected under sterile conditions. Once isolated cells are obtained, they are allowed to adhere to an adherent material (e.g., configured as a surface) to thereby isolate adherent cells. This may be effected prior to (see Example 1) or concomitant with culturing in 3D culturing conditions.

As used herein “an adherent material” refers to a synthetic, naturally occurring or a combination of same of a non-cytotoxic (i.e., biologically compatible) material having a chemical structure (e.g., charged surface exposed groups) which may retain the cells on a surface.

Examples of adherent materials which may be used in accordance with embodiment include, but are not limited to, a polyester, a polyalkylene, a polyfluorochloroethylene, a polyvinyl chloride, a polystyrene, a polysulfone, a cellulose acetate, a glass fiber, a ceramic particle, a matrigel, an extra cellular matrix component (e.g., fibronectin, chondronectin, laminin), a collagen, a poly L lactic acid and an inert metal fiber.

Further steps of purification or enrichment for stromal stem cells may be effected using methods which are well known in the art (such as by FACS using stromal stem cell marker expression, as further described herein below).

Non-limiting examples of base media useful in culturing include Minimum Essential Medium Eagle, ADC-I, LPM (Bovine Serum Albumin-free), F10(HAM), F12 (HAM), DCCMI, DCCM2, RPMI 1640, BGJ Medium (with and without Fitton-Jackson Modification), Basal Medium Eagle (BME—with the addition of Earle's salt base), Dulbecco's Modified Eagle Medium (DMEM-without serum), Yamane, IMEM-20, Glasgow Modification Eagle Medium (GMEM), Leibovitz L-15 Medium, McCoy's SA Medium, Medium M199 (M199E—with Earle's sale base), Medium M199 (M199H—with Hank's salt base), Minimum Essential Medium Eagle (MEM-E—with Earle's salt base), Minimum Essential Medium Eagle (MEM-H—with Hank's salt base) and Minimum Essential Medium Eagle (MEM-NAA with non essential amino acids), among numerous others, including medium 199, CMRL 1415, CMRL 1969, CMRL 1066, NCTC 135, MB 75261, MAB 8713, DM 145, Williams' G, Neuman & Tytell, Higuchi, MCDB 301, MCDB 202, MCDB 501, MCDB 401, MCDB 411, MDBC 153. A preferred medium for use is DMEM. These and other useful media are available from GIBCO, Grand Island, N.Y., USA and Biological Industries, Bet HaEmek, Israel, among others. A number of these media are summarized in Methods in Enzymology, Volume LVIII, “Cell Culture”, pp. 62 72, edited by William B. Jakoby and Ira H. Pastan, published by Academic Press, Inc.

The medium may be supplemented such as with serum such as fetal serum of bovine or other species, and optionally or alternatively, growth factors, cytokines, and hormones (e.g., growth hormone, erythropoeitin, thrombopoietin, interleukin 3, interleukin 6, interleukin 7, macrophage colony stimulating factor, c-kit ligand/stem cell factor, osteoprotegerin ligand, insulin, insulin like growth factors, epidermal growth factor, fibroblast growth factor, nerve growth factor, cilary neurotrophic factor, platelet derived growth factor, and bone morphogenetic protein at concentrations of between pigogram/ml to milligram/ml levels.

It is further recognized that additional components may be added to the culture medium. Such components may be antibiotics, antimycotics, albumin, amino acids, and other components known to the art for the culture of cells. Additionally, components may be added to enhance the differentiation process when needed (see further below).

Once adherent cells are at hand they may be passaged to three dimensional settings (see Example 1 of the Examples section which follows). It will be appreciated though, that the cells may be transferred to a 3D-configured matrix immediately after isolation (as mentioned hereinabove).

Thus, the adherent material of this embodiment is configured for 3D culturing thereby providing a growth matrix that substantially increases the available attachment surface for the adherence of the stromal cells so as to mimic the infrastructure of the tissue (e.g., placenta).

For example, for a growth matrix of 0.5 mm in height, the increase is by a factor of at least from 5 to 30 times, calculated by projection onto a base of the growth matrix. Such an increase by a factor of about 5 to 30 times, is per unit layer, and if a plurality of such layers, either stacked or separated by spacers or the like, is used, the factor of 5 to 30 times applies per each such structure. When the matrix is used in sheet form, preferably non-woven fiber sheets, or sheets of open-pore foamed polymers, the preferred thickness of the sheet is about 50 to 1000 μm or more, there being provided adequate porosity for cell entrance, entrance of nutrients and for removal of waste products from the sheet. According to a preferred embodiment the pores have an effective diameter of 10 μm to 100 μm. Such sheets can be prepared from fibers of various thicknesses, the preferred fiber thickness or fiber diameter range being from about 0.5 μm to 20 μm, still more preferred fibers are in the range of 10 μm to 15 μm in diameter.

The described structures may be supported by, or even better bonded to, a porous support sheet or screen providing for dimensional stability and physical strength.

Such matrix sheets may also be cut, punched, or shredded to provide particles with projected area of the order of about 0.2 mm² to about 10 mm², with the same order of thickness (about 50 to 1000 μm).

Further details relating to the fabrication, use and/or advantages of the growth matrix which was used in the described reduction to practice are described in U.S. Pat. No. 5,168,085, and in particular, U.S. Pat. No. 5,266,476, both are incorporated herein by reference.

The adherent surface may have a shape selected from the group consisting of squares, rings, discs, and cruciforms.

For high scale production, culturing is preferably effected in a 3D bioreactor.

Examples of such bioreactors include, but are not limited to, a plug flow bioreactor, a continuous stirred tank bioreactor and a stationary-bed bioreactor.

As shown Example 1 of the Examples section, a three dimensional (3D) plug flow bioreactor (as described in U.S. Pat. No. 6,911,201) is capable of supporting the growth and prolonged maintenance of stromal cells. In this bioreactor, stromal cells are seeded on porrosive carriers made of a non woven fabric matrix of polyester, packed in a glass column, thereby enabling the propagation of large cell numbers in a relatively small volume.

The matrix used in the plug flow bioreactor can be of sheet form, non-woven fiber sheets, or sheets of open-pore foamed polymers, the preferred thickness of the sheet is about 50 to 1000 μm or more, there being provided adequate porosity for cell entrance, entrance of nutrients and for removal of waste products from the sheet.

Other 3D bioreactors that can be used include, but are not limited to, a continuous stirred tank bioreactor, where a culture medium is continuously fed into the bioreactor and a product is continuously drawn out, to maintain a time-constant steady state within the reactor]. A stirred tank bioreactor with a fibrous bed basket is available for example at New Brunswick Scientific Co., Edison, N.J.), A stationary-bed bioreactor, an air-lift bioreactor, where air is typically fed into the bottom of a central draught tube flowing up while forming bubbles, and disengaging exhaust gas at the top of the column], a cell seeding perfusion bioreactor with Polyactive foams [as described in Wendt, D. et al., Biotechnol Bioeng 84: 205-214, (2003)] tubular poly-L-lactic acid (PLLA) porous scaffolds in a Radial-flow perfusion bioreactor [as described in Kitagawa et al., Biotechnology and Bioengineering 93(5): 947-954 (2006). Other bioreactors which can be used are described in U.S. Pat. Nos. 6,277,151, 6,197,575, 6,139,578, 6,132,463, 5,902,741 and 5,629,186.

Cell seeding is preferably effected 100,000-1,500,000 cells/mm at seeding.

Cells are preferably harvested once reaching at least about 40% confluence, 60% confluence or 80% confluence while preferably avoiding uncontrolled differentiation and senescence.

Culturing is effected for at least about 2 days, 3 days, 5 days, 10 days, 20 days, a month or even more. It will be appreciated that culturing in a bioreactor may prolong this period. Passaging may also be effected to increase cell number.

The described adherent cells preferably comprise at least one “stromal stem cell phenotype”.

As used herein “a stromal stem cell phenotype” refers to a structural or functional phenotype typical of a bone-marrow derived stromal (i.e., mesenchymal) stem cell

As used herein the phrase “stem cell” refers to a cell which is not terminally differentiated.

Thus for example, the cells may have a spindle shape. Alternatively or additionally the cells may express a marker or a collection of markers (e.g. surface marker) typical to stromal stem cells. Examples of stromal stem cell surface markers (positive and negative) include but are not limited to CD105+, CD29+, CD44+, CD73+, CD90+, CD34−, CD45−, CD80−, CD19−, CD5−, CD20−, CD11B−, CD14−, CD19−, CD79−, HLA-DR−, and FMC7−. Other stromal stem cell markers include but are not limited to tyrosine hydroxylase, nestin and H—NF.

Examples of functional phenotypes typical of stromal stem cells include, but are not limited to, T cell suppression activity (don't stimulate T cells and conversely suppress same), hematopoietic stem cell support activity, as well as adipogenic, hepatogenic, osteogenic and neurogenic differentiation.

Any of these structural or functional features can be used to qualify the described cells (see Examples 1-2 of the Examples section which follows).

Populations of cells generated according to the present teachings are characterized by a unique protein expression profile as is shown in Example 1 of the Examples section. Thus for example, adherent cells of placenta or adipose tissue generated according to the present teachings, are capable of expressing and/or secreting high levels of selected factors. For example, such cells express or secrete SCF, Flt-3, H2AF or ALDH X at least 2, 3, 4, 5, 6, 7, 8, 9, 10, 11 or preferably 12 fold higher than that expressed or secreted by adherent cells of placenta or adipose tissue grown in a 2D culture. Additionally or alternatively, the described population of cells secrete or express IL-6, EEEF2, RCN2 or CNN1 at a level least 2, 3 or 5 fold higher than that expressed or secreted by adherent cells of placenta or adipose tissue grown in a 2D culture. Additionally or alternatively, the described population of cells are characterized by lower level of expression of various other proteins as compared to 2D cultured cells. Thus for example, secrete or express less than 0.6, 0.5, 0.25 or 0.125 of the expression level of Hnrphl1, CD44 antigen isoform 2 precursor, Papss2 or rpL7a expressed or secreted by adherent cells of placenta or adipose tissue grown in a 2D culture.

Furthermore, the present inventors have realized that adherent stromal cells, and particularly 3D-ASCs, showed immunosuppressive activity. As is shown in Example 3 of the Examples section which follows, adherent stromal cells, and particularly 3D-ASCs, were found to suppress the immune reaction of human cord blood mononuclear cells in an MLR assay. Thus, the described cells may comprise biological activities which may be preferentially used in the clinic (e.g., T cell suppression activity, hematopoietic stem cell support activity).

Furthermore, the present inventors have realized that conditioned medium of the described cells may comprise biological activities which may be preferentially used in the clinic (e.g., T cell suppression activity, hematopoietic stem cell support activity).

Thus, there is further envisaged collection of conditioned medium and its use as is or following further steps of concentration, enrichment or fractionation using methods which are well known in the art. Preferably a conditioned medium of the present is obtained from a high viability mid-log culture of cells.

As mentioned hereinabove, the described cells and conditioned media are characterized by a stromal stem cell phenotype and as such can be used in any research and clinical application which may benefit from the use of such cells.

Engraftment and initiation of hematopoiesis by transplanted HSCs depend on complex processes which include homing, following a gradient of chemokines across the endothelial cell barrier, to the bone marrow and lodging in the appropriate niches, while establishing physical contacts between transplanted cells, the ECM and the mesenchymal cells of the niches. All these processes involve a complex array of molecules, such as cytokines, hormones, steroids, extra cellular matrix proteins, growth factors, cell-to-cell interaction and adhesion proteins, and matrix proteins.

It is known that only 1-5% of transfused HSCs are detected in the recipient BM 2-3 days post transplantation [Kerre et al., J Immunol. 167:3692-8. (2001); Jetmore et al., Blood. 99:1585-93 (2002)].

MSCs contribution to hematopoietic engraftment is in part by the inhibition of donor derived T cell production, which cause graft vs. host disease [GvHD, Charbord P., and Moore, K., Ann. N Y. Acad. Sci. 1044: 159-167 (2005); Maitra B, et al., Bone Marrow Transplant. 33(6):597-604. (2004); U.S. Pat. Nos. 6,010,696; 6,555,374]; and part by providing a hematopoietic stem cell (HSC) support (i.e., sustaining and aiding the proliferation, maturation and/or homing of hematopoietic stem cells).

As shown in Example 2 of the Examples section which follows, placenta and adipose tissue-derived adherent cells were surprisingly found to be supportive of HSC engraftment even after chemotherapy.

Given these results it is conceivable that the described cells or media may be used in any clinical application for which stromal stem cell transplantation is used.

Thus, according to another embodiment, there is provided a method of treating a medical condition (e.g., pathology, disease, syndrome) which may benefit from stromal stem cell transplantation in a subject in need thereof.

As used herein the term “treating” refers to inhibiting or arresting the development of a pathology and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology. Preferably, the term “treating” refers to alleviating or diminishing a symptom associated with a cancerous disease. Preferably, treating cures, e.g., substantially eliminates, the symptoms associated with the medical condition.

As used herein “a medical condition which may benefit from stromal stem cell transplantation” refers to any medical condition which may be alleviated by administration of the described cells/media.

The term or phrase “transplantation”, “cell replacement” or “grafting” are used interchangeably herein and refer to the introduction of the described cells to target tissue.

As used herein the term “subject” refers to any subject (e.g., mammal), preferably a human subject.

The method of this embodiment comprises administering to the subject a therapeutically effective amount of the described cells or media (described hereinabove), thereby treating the medical condition which may benefit from stromal stem cell transplantation in the subject

Cells which may be administered in accordance with this embodiment include the above-described adherent cells which may be cultured in either 2D or 3D settings as well as mesenchymal and non mesenchymal partially or terminally differentiated derivatives of same.

Methods of deriving lineage specific cells from the described stromal stem cells are well known in the art. See for example, U.S. Pat. Nos. 5,486,359, 5,942,225, 5,736,396, 5,908,784 and 5,902,741.

The cells may be naïve or genetically modified such as to derive a lineage of interest (see U.S. Pat. Appl. No. 20030219423).

The cells and media may be of autologous or non-autologous source (i.e., allogenic or xenogenic) of fresh or frozen (e.g., cryo-preserved) preparations.

Depending on the medical condition, the subject may be administered with additional chemical drugs (e.g., immunomodulatory, chemotherapy etc.) or cells.

Thus, for example, for improving stem cell engraftment (e.g., increasing the number of viable HSC in the recipient BM and optimally improve normal white blood cell count) the described cells/media may be administered prior to, concomitantly with or following HSC transplantation.

Preferably the HSCs and stromal cells share common HLA antigens. Preferably, the HSCs and stromal cells are from a single individual. Alternatively, the HSCs and stromal cells are from different individuals.

The term or phrase “transplantation”, “cell replacement” or “grafting” are used interchangeably herein and refer to the introduction of the described cells to target tissue. The cells can be derived from the recipient or from an allogeneic or xenogeneic donor.

Since non-autologous cells are likely to induce an immune reaction when administered to the body several approaches have been developed to reduce the likelihood of rejection of non-autologous cells. These include either suppressing the recipient immune system or encapsulating the non-autologous cells in immunoisolating, semipermeable membranes before transplantation.

Encapsulation techniques are generally classified as microencapsulation, involving small spherical vehicles and macroencapsulation, involving larger flat-sheet and hollow-fiber membranes (Uludag, H. et al. Technology of mammalian cell encapsulation. Adv Drug Deliv Rev. 2000; 42: 29-64).

Methods of preparing microcapsules are known in the arts and include for example those disclosed by Lu M Z, et al., Cell encapsulation with alginate and alpha-phenoxycinnamylidene-acetylated poly(allylamine). Biotechnol Bioeng. 2000, 70:479-83, Chang T M and Prakash S. Procedures for microencapsulation of enzymes, cells and genetically engineered microorganisms. Mol Biotechnol. 2001, 17: 249-60, and Lu M Z, et al., A novel cell encapsulation method using photosensitive poly(allylamine alpha-cyanocinnamylideneacetate). J Microencapsul. 2000, 17: 245-51.

For example, microcapsules are prepared by complexing modified collagen with a ter-polymer shell of 2-hydroxyethyl methylacrylate (HEMA), methacrylic acid (MAA) and methyl methacrylate (MMA), resulting in a capsule thickness of 2-5 μm. Such microcapsules can be further encapsulated with additional 2-5 μm ter-polymer shells in order to impart a negatively charged smooth surface and to minimize plasma protein absorption (Chia, S. M. et al. Multi-layered microcapsules for cell encapsulation Biomaterials. 2002 23: 849-56).

Other microcapsules are based on alginate, a marine polysaccharide (Sambanis, A. Encapsulated islets in diabetes treatment. Diabetes Technol. Ther. 2003, 5: 665-8) or its derivatives. For example, microcapsules can be prepared by the polyelectrolyte complexation between the polyanions sodium alginate and sodium cellulose sulphate with the polycation poly(methylene-co-guanidine) hydrochloride in the presence of calcium chloride.

It will be appreciated that cell encapsulation is improved when smaller capsules are used. Thus, the quality control, mechanical stability, diffusion properties, and in vitro activities of encapsulated cells improved when the capsule size was reduced from 1 mm to 400 μm (Canaple L. et al., Improving cell encapsulation through size control. J Biomater Sci Polym Ed. 2002; 13:783-96). Moreover, nanoporous biocapsules with well-controlled pore size as small as 7 nm, tailored surface chemistries and precise microarchitectures were found to successfully immunoisolate microenvironments for cells (Williams D. Small is beautiful: microparticle and nanoparticle technology in medical devices. Med Device Technol. 1999, 10: 6-9; Desai, T. A. Microfabrication technology for pancreatic cell encapsulation. Expert Opin Biol Ther. 2002, 2: 633-46).

Examples of immunosuppressive agents include, but are not limited to, methotrexate, cyclophosphamide, cyclosporine, cyclosporin A, chloroquine, hydroxychloroquine, sulfasalazine (sulphasalazopyrine), gold salts, D-penicillamine, leflunomide, azathioprine, anakinra, infliximab (REMICADE), etanercept, TNF.alpha. blockers, a biological agent that targets an inflammatory cytokine, and Non-Steroidal Anti-Inflammatory Drug (NSAIDs). Examples of NSAIDs include, but are not limited to acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors and tramadol.

In any of the methods described herein, the cells or media can be administered either per se or, preferably as a part of a pharmaceutical composition that further comprises a pharmaceutically acceptable carrier.

As used herein a “pharmaceutical composition” refers to a preparation of one or more of the chemical conjugates described herein, with other chemical components such as pharmaceutically suitable carriers and excipients. The purpose of a pharmaceutical composition is to facilitate administration of a compound to a subject.

Hereinafter, the term “pharmaceutically acceptable carrier” refers to a carrier or a diluent that does not cause significant irritation to a subject and does not abrogate the biological activity and properties of the administered compound. Examples, without limitations, of carriers are propylene glycol, saline, emulsions and mixtures of organic solvents with water.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

According to certain embodiments, the pharmaceutical carrier is an aqueous solution of saline.

Techniques for formulation and administration of drugs may be found in “Remington's Pharmaceutical Sciences,” Mack Publishing Co., Easton, Pa., latest edition, which is incorporated herein by reference.

One may administer the pharmaceutical composition in a systemic manner (as detailed hereinabove). Alternatively, one may administer the pharmaceutical composition locally, for example, via injection of the pharmaceutical composition directly into a tissue region of a patient.

The described pharmaceutical compositions may be manufactured by processes well known in the art, e.g., by means of conventional mixing, dissolving, granulating, dragee-making, levigating, emulsifying, encapsulating, entrapping or lyophilizing processes.

Pharmaceutical compositions for use thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For any preparation used in the described methods, the therapeutically effective amount or dose can be estimated initially from in vitro and cell culture assays. Preferably, a dose is formulated in an animal model to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals.

The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition, (see e.g., Fingl, et al., 1975, in “The Pharmacological Basis of Therapeutics”, Ch. 1 p. 1).

For injection, the active ingredients of the pharmaceutical composition may be formulated in aqueous solutions, preferably in physiologically compatible buffers such as Hank's solution, Ringer's solution; or physiological salt buffer.

Dosage amount and interval may be adjusted individually to levels of the active ingredient which are sufficient to effectively regulate the neurotransmitter synthesis by the implanted cells. Dosages necessary to achieve the desired effect will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or diminution of the disease state is achieved.

The amount of a composition to be administered will, of course, be dependent on the individual being treated, the severity of the affliction, the manner of administration, the judgment of the prescribing physician, etc. The dosage and timing of administration will be responsive to a careful and continuous monitoring of the individual changing condition.

Following transplantation, the described cells preferably survive in the diseased area for a period of time (e.g. at least 6 months), such that a therapeutic effect is observed.

Compositions including the described preparation formulated in a compatible pharmaceutical carrier may also be prepared, placed in an appropriate container, and labeled for treatment of an indicated condition.

The described compositions may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S; Food and Drug Administration for prescription drugs or of an approved product insert.

### EXAMPLES

### Example 1

### Production and Culturing of Adherent Stromal Cells (ASC) from Bone Marrow, Placenta and Adipose Tissues

Adherent cells were cultured in a bioreactor system containing 3D carriers to produce 3D-ASC cells, characterized by a specific cell marker expression profile. Growth efficiency was tested through cell count.

### Materials and Experimental Procedures

Bone marrow stromal cells—Bone marrow (BM) stromal cells were obtained from aspirated sterna marrow of hematologically healthy donors undergoing open-heart surgery or BM biopsy. Marrow aspirates were diluted 3-fold in Hank's Balanced Salts Solution (HBSS; GIBCO BRL/Invitrogen, Gaithersburg Md.) and subjected to Ficoll-Hypaque (Robbins Scientific Corp. Sunnyvale, Calif.) density gradient centrifugation. Thereafter, marrow mononuclear cells (less than 1.077 gm/cm³) were collected, washed 3 times in HBSS and resuspended in growth media [DMEM (Biological Industries, Beit Ha'emek, Israel) supplemented with 10% FCS (GIBCO BRL), 10⁻⁴ M mercaptoethanol (Merck, White House Station, N.J.), Pen-Strep-Nystatin mixture (100 U/ml:100 mg/ml:1.25 μg/ml; Beit Ha'Emek), 2 mM L-glutamine (Beit Ha'Emek)]. Cells from individual donors were incubated separately in tissue culture flasks (Corning, Acton, Mass.) at 37° centigrade (5% CO₂) with weekly change of culture media. Cells were split every 3-4 days using 0.25 percent trypsin-EDTA (Beit Ha'Emek). Following 2-40 passages, when reaching 60-80 percent confluence, cells were collected for analysis or for culturing in bioreactors.

Placenta derived stromal cells—Inner parts of a full-term delivery placenta (Bnei Zion medical center, Haifa, Israel) were cut under sterile conditions, washed 3 times with Hank's Buffer and incubated for 3 hours at 37° C. with 0.1% Collagenase (1 mg/ml tissue; Sigma-Aldrich, St. Lewis, Mo.). Using gentle pipetting, suspended cells were then washed with DMEM supplemented with 10% FCS, Pen-Strep-Nystatin mixture (100 U/ml:100 μg/ml:1.25 un/ml) and 2 mM L-glutamine, seeded in 75 cm² flasks and incubated at 37° C. in a tissue culture incubator under humidified condition with 5% CO₂ Thereafter, cells were allowed to adhere to a plastic surface for 72 hours after which the media was changed every 3-4 days. When reaching 60-80% confluence cells were detached from the growth flask using 0.25% trypsin-EDTA and seeded into new flasks. Cultured cells were thereafter collected for analysis or for culturing in bioreactors.

Adipose derived stromal cells—Stromal cells were obtained from human adipose tissue of liposuction procedures (Rambam Haifa, Israel). Adipose tissue was washed extensively with equal volumes of PBS and digested at 37° centigrade for 30 min with collagenase (20 mg/ml). Cells were then washed with DMEM containing 10% FCS, Pen-Strep-Nystatin mixture (100 U/ml:100 μg/ml:1.25 un/ml) and L-Glutamin and centrifuged at 1200 rpm for 10 min RT, resuspended with lysing solution (1:10; Biological Industries, Beit Ha'emek, Israel, in order to discard red-blood cells) centrifuged and resuspended with DMEM containing 10% FCS, Pen-Strep-Nystatin mixture (100 U/ml:100 μg/ml:1.25 un/ml) and L-Glutamine. Washed cells were then seeded in a sterile tissue culture medium flask at 3-10×10⁷ cells/flask. At the next day cells were washed with PBS to remove residual RBC and dead cells. The cells were kept at 37° centigrade in a tissue culture incubator under humidified condition with 5% CO₂. The medium was changed every 3 to 4 days. At 60-80% confluence, the cells were detached from the growth flask using 0.25% trypsin-EDTA and seeded into new flasks. Following 2-40 passages, when cells reached 60-80% confluence, cells were collected for analysis or for culturing in bioreactors.

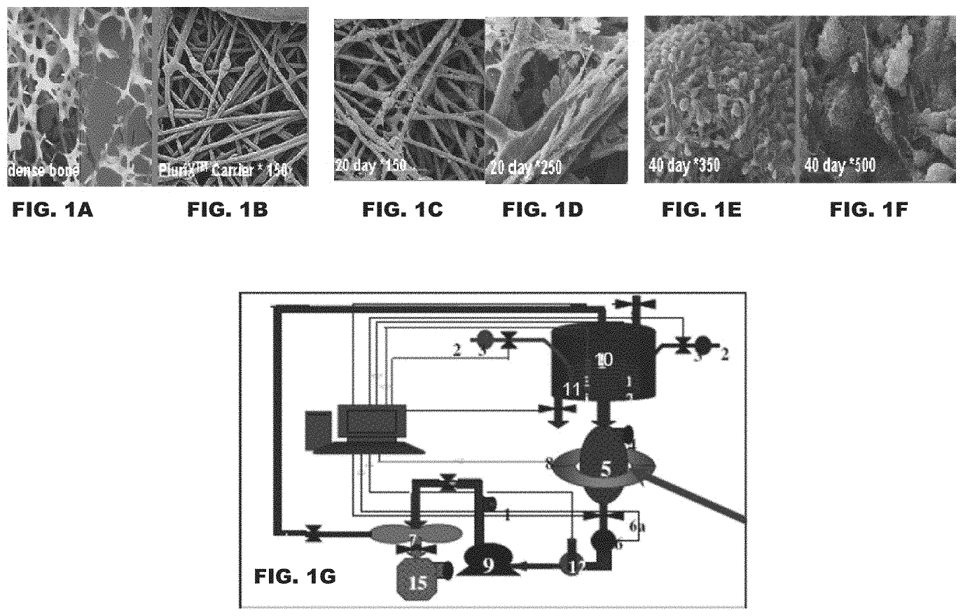

PluriX™ Plug Flow bioreactor—The PluriX™ Plug Flow bioreactor (Pluristem, Haifa, Israel; as illustrated in FIG. 1G, was loaded with 1-100 ml packed 3D porrosive carriers (4 mm in diameter) made of a non-woven fabric matrix of polyester. These carriers enable the propagation of large cell numbers in a relatively small volume. Glassware was designed and manufactured by Pluristem (Pluristem, Haifa, Israel). The bioreactor was maintained in an incubator of 37° C., with flow rate regulated and monitored by a valve (6a), and peristaltic pump (9). The bioreactor contains a sampling and injection point (4), allowing the sequential seeding of cells. Culture medium was supplied at pH 6.7-7.4 from a reservoir (1). The reservoir was supplied by a filtered gas mixture (2,3), containing air/CO₂/O₂ at differing proportions, depending on cell density in the bioreactor. The 02 proportion was suited to the level of dissolved 02 at the bioreactor exit, determined by a monitor (6). The gas mixture was supplied to the reservoir via silicone tubes or diffuser (Degania Bet, Emek Hayarden, Israel). The culture medium was passed through a separating container (7) which enables collection of circulating, non-adherent cells. Circulation of the medium was obtained by a peristaltic pump (9). The bioreactor was further equipped with an additional sampling point (10) and containers for continuous medium exchange.

Production of 3D-adherent stromal cells (3D-ASC)—Non-confluent primary human adherent 2D cell cultures, grown as described above, were trypsinized, washed, resuspended in DMEM supplemented with 10% FBS, Pen-Strep-Nystatin mixture (100 U/ml:100 μlg/ml:1.25 un/ml) and 2 mM L-glutamine, and seeded (10³-10⁵ cells/ml) via an injection point onto the 3D carriers in a sterile Plug Flow bioreactor (see FIG. 1G). Prior to inoculation, bioreactor was filled with PBS—Ca—Mg (Biological Industries, Beit Ha'emek, Israel), autoclaved (120° C., 30 min) and washed with Dulbecco's growth medium containing 10% heat-inactivated fetal calf serum and a Pen-Strep-Nystatin mixture (100 U/ml: 100 μg/ml: 1.25 un/ml). Flow was kept at a rate of 0.1-5 ml/min. Seeding process involved cease of circulation for 2-48 hrs, thereby allowing the cells to settle on the carriers. Bioreactor was kept under controlled temperature (37° C.) and pH conditions (pH=6.7-7.4); using an incubator supplied with sterile air and CO₂ as needed. Growth medium was replaced 2-3 times a week. Circulation medium was replaced with fresh DMEM media, every 4 hr to 7 days. At a density of 1×10⁶-1×10⁷ cells/ml (following 12-40 days of growth), total medium volume was removed from the bioreactor and bioreactor and carriers were washed 3-5 times with PBS. 3D-ASC cells were then detached from the carriers with Trypsin-EDTA; (Biological Industries, Beit Ha'emek, Israel; 3-15 minutes with gentle agitation, 1-5 times), and were thereafter resuspended in DMEM and cryopreserved.

3D-ASC quality biological assays—Cryopreserved 3D-ASC cells were thawed and counted. For cell viability evaluation, 2×10⁵ cells were seeded in a 150 cm² tissue culture flask and their adherence capability and repopulation was evaluated within 7 days following seeding. Thereafter, the 3D-ASC membrane marker phenotype was analyzed using fluorescence monoclonal antibodies flow-cytometer (Beckman Coulter, Fullerton, Calif.).

Comparison between the cell membrane marker profile of 3D and 2D cultured adherent cells using flow cytometry assays—100,000-200,000 adherent cells from 2D cultures and 3D flow system cultures were suspended in 0.1 ml of culture medium in a 5 ml tube and incubated (4° centigrade, 30 min, dark conditions) with saturating concentrations of each of the following MAbs: FITC-conjugated anti-human CD90 (Chemicon International Inc. Temecula, Calif.), PE-conjugated anti-human CD73 (Bactlab Diagnostic, Ceasarea, Israel), PE-conjugated anti-human CD105 (eBioscience, San Diego, Calif.), FITC-conjugated anti-human CD29 (eBioscience, San Diego, Calif.), Cy7-PE conjugated anti-human CD45 (eBiosience), PE-conjugated anti-human CD19 (IQProducts, Groningen, The Netherlands), PE-conjugated anti-human CD14 MAb (IQProducts), FITC-conjugated anti-human CD11b (IQProducts) and PE-conjugated anti-human CD34 (IQProducts) or with FITC-conjugated anti-human HLA-DR MAb (IQProducts). Following incubation the cells were washed twice in ice-cold PBS containing 1 percent heat-inactivated FCS, resuspended in 500 μl formaldehyde 0.5 percent and analyzed using the FC-500 flow-cytometer (Beckman Coulter, Fullerton, Calif.).

Comparison between the protein profile of 3D and 2D cultured adherent cells using mass spectrometry analysis—2D and 3D derived culturing procedures ASCs were produced from the placenta as described above. Briefly, the 2D cultures were produced by culturing 0.3-0.75×10⁶ cells in 175 cm² flasks for 4 days under humidified 5% CO₂ atmosphere at 37° centigrade, until reaching 60-80% confluence. The 3D cultures were produced by seeding 2-10×10⁶ cells/gram in a bioreactor containing 2000 carriers, and culturing for 18 days. Following harvesting, cells were washed (×3) to remove all the serum, pelleted and frozen. Proteins were isolated from pellets [using Tri Reagent kit (Sigma, Saint Louis, USA) and digested with trypsin and labeled with iTRAQ reagent (Applied Biosciences, Foster City, Calif.)], according to the manufacturers protocol. Briefly, iTRAQ reagents are non-polymeric, isobaric tagging reagents. Peptides within each sample are labeled with one of four isobaric, isotope-coded tags via their N-terminal and/or lysine side chains. The four labeled samples are mixed and peptides are analyzed with mass spectrometery. Upon peptide fragmentation, each tag releases a distinct mass reporter ion; the ratio of the four reporters therefore gives relative abundances of the given peptide in a sample, (information at: http://docs.appliedbiosystems.com/pebiodocs/00113379.pdf).

Proteomics analysis of 2D culture versus 3D culture of placenta derived ASCs was performed in the Smoler proteomic center (Department of Biology, Technion, Haifa, Israel) using LC-MS/MS on QTOF-Premier (Waters, San Francisco, Calif.), with identification and analysis done by Pep-Miner software [Beer, I., Proteomics, 4, 950-60 (2004)] against the human part of the nr database. The proteins analyzed were: heterogeneous nuclear ribonucleoprotein H1 (Hnrph1 GeneBank Accession No. NP_005511), H2A histone family (H2AF, GeneBank Accession No. NP_034566.1), eukaryotic translation elongation factor 2 (EEEF2, GeneBank Accession No. NP_031933.1), reticulocalbin 3, EF-hand calcium binding domain (RCN2, GeneBank Accession No. NP 065701), CD44 antigen isoform 2 precursor (GeneBank Accession No. NP OO 1001389, calponin 1 basic smooth muscle (CNN1, GeneBank Accession No. NP_001290), 3 phosphoadenosine 5 phosphosulfate synthase 2 isoform a (Papss2, GeneBank Accession No. NP 004661), ribosomal protein L7a (rpL7a, GeneBank Accession No. NP_000963) and Aldehyde dehydrogenase X (ALDH X, GeneBank Accession No. P47738). Every experiment was done twice. Because of the nature of the analysis, every protein was analyzed according to the number of peptides of which appeared in a sample (2-20 appearances of a protein in each analysis)

Comparison between secreted proteins in 3D and 2D cultured adherent cells using ELISA—2D and 3D derived culturing procedures ASCs produced from the placenta, were produced as described above, with 3D cultures for the duration of 24 days. Conditioned media were thereafter collected and analyzed for Flt-3 ligand, IL-6, Trombopoietin (TPO) and stem cell factor (SCF), using ELISA (R and D Systems, Minneapolis, Minn.), in three independent experiments. Results were normalized for 1×10⁶ cells/ml.

### Results

### The PluriX™ Bioreactor System Creates a Physiological-Like Microenvironment.

In order to render efficient culture conditions for adherent cells, a physiological-like environment (depicted in FIG. 1A) was created artificially, using the PluriX Bioreactor (Pluristem, Haifa, Israel; carrier is illustrated in FIG. 1G and shown before seeding in FIG. 1B). As is shown in FIGS. 1C-F, bone marrow produced 3D-ASC cells were cultured successfully and expanded on the 3D matrix, 20 days (FIGS. 1B-C, magnified ×150 and 250 respectively) and 40 days (FIGS. 1C-D, magnified ×350 and 500 respectively) following seeding.

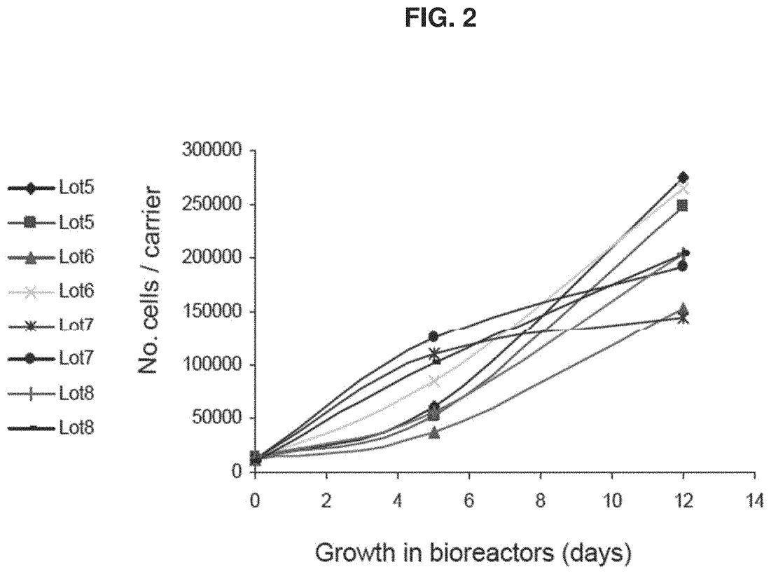

Cells grown in the PluriX Bioreactor system were significantly expanded—Different production lots of placenta-derived 3D-ASC cells were grown in the PluriX bioreactor systems. The seeding density was 13,300 cells/carrier (to a total of 2×10⁶ cells). Fourteen days following seeding, cell density multiplied by 15 fold, reaching approximately 200,000 cells/carrier (FIG. 2), or 30×10⁶ in a bioreactor of 150 carriers. In a different experiment, cells were seeded into the bioreactor at density of 1.5×10⁴ cells/ml and 30 days following seeding the carriers contained an over 50-fold higher cell number, i.e. approx. 0.5×10⁶ cells/carrier, or 0.5×10⁷ cells/ml. The cellular density on the carriers at various levels of the growth column was consistent, indicating a homogenous transfer of oxygen and nutrients to the cells. The 3D culture system was thus proven to provide supporting conditions for the growth and prolonged maintenance of high-density mesenchymal cells cultures, which can be grown efficiently to an amount sufficient for the purpose of supporting engraftment and successful transplantation.

3D-ASCs show unique membrane marker characteristics—In order to define the difference in the secretion profile of soluble molecules and protein production, effected by the bone environment mimicking 3D culturing procedure, FACs analysis was effected. As is shown in FIG. 3A, FACS analysis of cell markers depict that 3D-ASCs display a different marker expression pattern than adherent cells grown in 2D conditions. 2D-cultured cells expressed significantly higher levels of positive membrane markers CD90, CD 105, CD73 and CD29 membrane markers as compared to 3D-cultured cells. For example, CD105 showed a 56% expression in 3D cultured cells vs. 87% in 2D cultured cells. ASCs of both 2D and 3D placenta cultures, did not express any hematopoietic membrane markers (FIG. 3B).

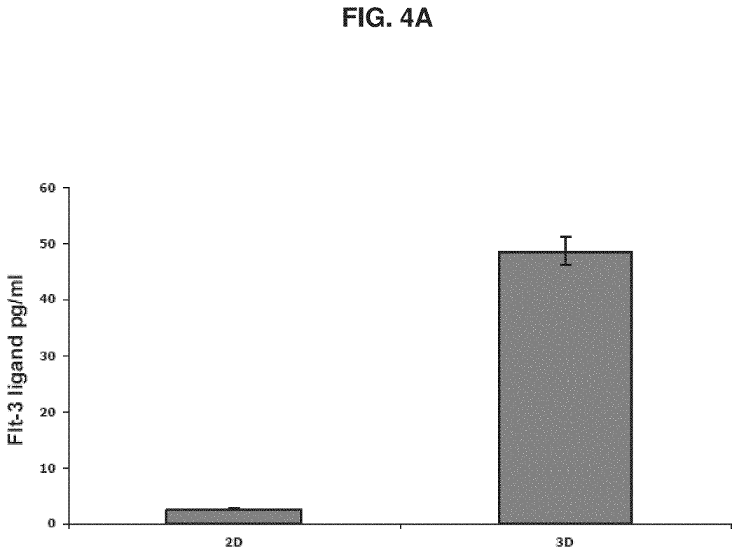

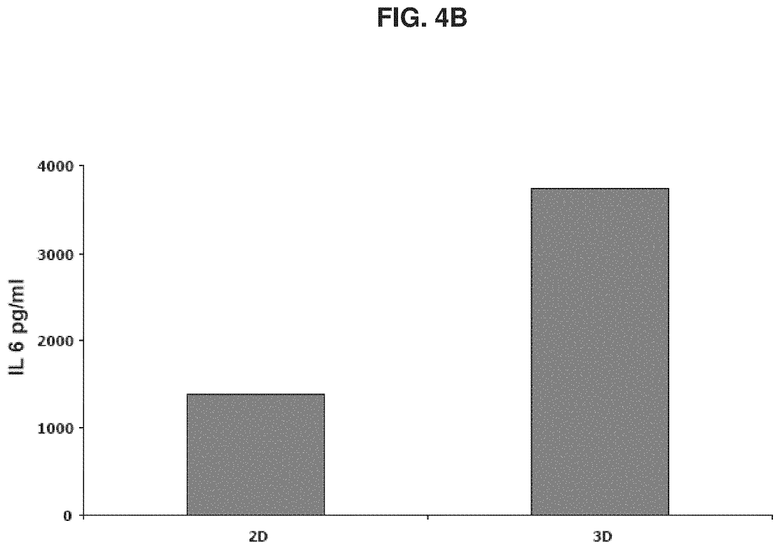

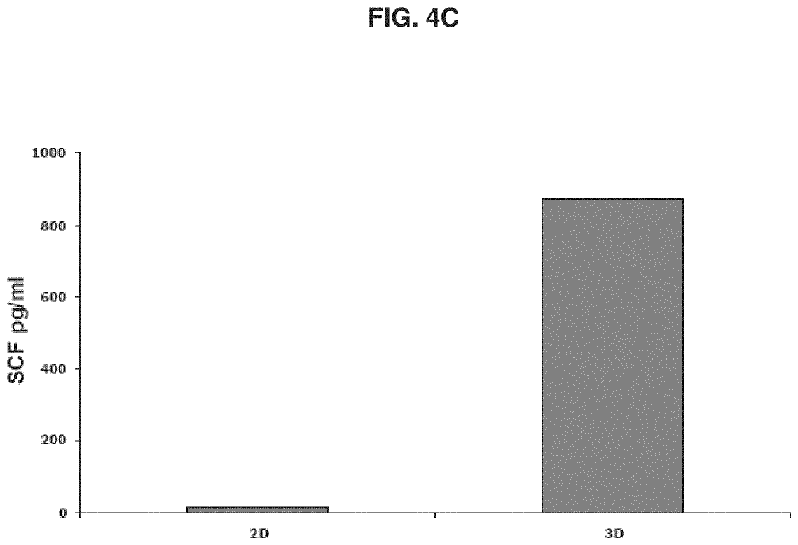

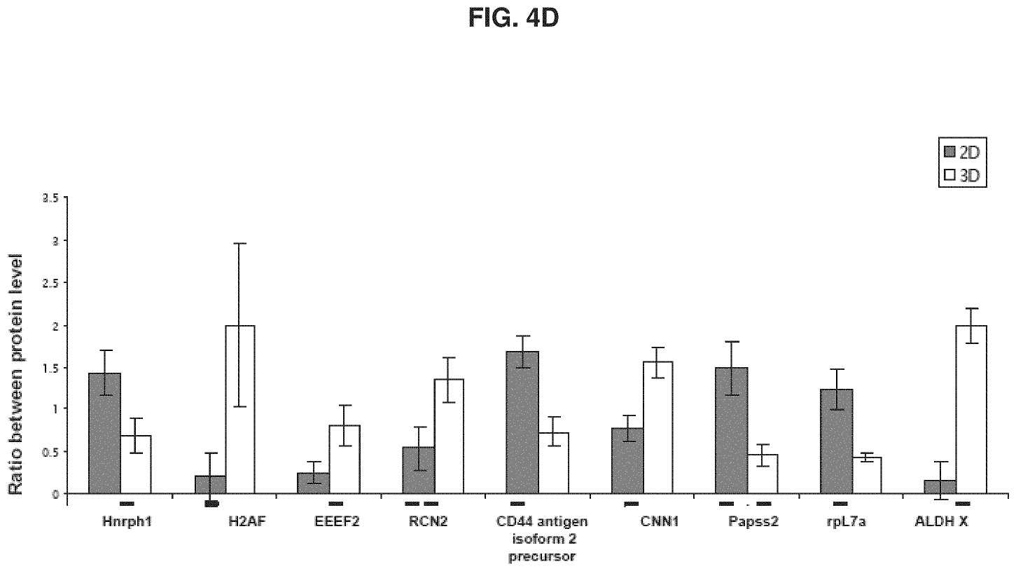

3D-ASCs show a unique profile of soluble factors—The hematopoietic niche includes supporter cells that produce an abundance of cytokines, chemokines and growth factors. In order to further define the difference between 2D and 3D cultured ASCs, the profile of the four main hematopoietic secreted proteins in the conditioned media of 2D and 3D ASC cultures was effected by ELISA. FIGS. 4A-C show that cells grown in 3D conditions produced conditioned media with higher levels of Flt-3 ligand (FIG. 4A), IL-6 (FIG. 4B), and SCF (FIG. 4C), while low levels of IL-6, and close to zero level of Flt-3 ligand and SCF, were detected in the condition media of 2D cultures. Production of Trombopoietin (TPO) was very low and equal in both cultures.

3D-ASCs show a unique protein profile in mass spectrometry analysis—In order to further define the difference between 2D and 3D cultured ASCs, the protein profile of these cells was analyzed by mass spectrometry. FIG. 4D shows that 2D and 3D cultured ASCs show a remarkably different protein expression profile. As is shown in Table 1 below, 3D cultured cells show a much higher expression level of H2AF and ALDH X (more than 9 and 12 fold higher, respectively) and a higher level of the proteins EEEF2, RCN2 and CNN1 (ca. 3, 2.5 and 2 fold, respectively). In addition, 3D cultured cells show ca. half the expression levels of the proteins Hnrph1 and CD44 antigen isoform 2 precursor and ca. a third of the expression levels of Papss2 and rpL7a.

TABLE 1

| | Protein level (relative to iTRAQ reporter group) | | | |
|---|---|---|---|---|
| | 3D cultured ASCs | | 2D cultured ASCs | |
| protein | Av | SD | Av | SD |
| Hnrph1 | 1.434493 | 0.260914 | 0.684687 | 0.197928 |
| H2AF | 0.203687 | 0.288058 | 1.999877 | 0.965915 |
| EEEF2 | 0.253409 | 0.130064 | 0.799276 | 0.243066 |
| RCN2 | 0.54 | 0.25 | 1.34 | 0.26 |
| CD44 | 1.68 | 0.19 | 0.73 | 0.17 |
| antigen isoform | | | | |
| 2 precursor | | | | |
| CNN1 | 0.77 | 0.15 | 1.55 | 0.17 |
| Papss2 | 1.48352 | 0.314467 | 0.45627 | 0.137353 |
| rpL7a | 1.22 | 0.24 | 0.43 | 0.05 |
| ALDH X | 0.15847 | 0.22411 | 1.98671 | 0.212851 |

### Example 2

### Assessment of the Ability of Placenta Derived 3D-ASC to Improve HSC Engraftment

3D-ASC support of HSC engraftment was evaluated by the level of human hematopoietic cells (hCD45+) detected in sub lethally irradiated or chemotherapy pretreated immune deficient NOD-SCID mice.

### Materials and Experimental Procedures

Isolation of CD34+ Cells—Umbilical cord blood samples were taken under sterile conditions during delivery (Bnei Zion Medical Center, Haifa, Israel) and mononuclear cells were fractionated using Lymphoprep (Axis-Shield PoC As, Oslo, Norway) density gradient centrifugation and were cryopreserved. Thawed mononuclear cells were washed and incubated with anti-CD34 antibodies and isolated using midi MACS (Miltenyl Biotech, Bergish Gladbach, Germany). Cells from more than one sample were pooled for achieving the desired amount (50,000-100,000 cells).

Detection of transplanted cells in irradiated mice—Seven week old male and female NOD-SCID mice (NOD-CB 17-Prkdcscid/J; Harlan/Weizmann Inst., Rehovot Israel) were maintained in sterile open system cages, given sterile diets and autoclaved acidic water. The mice were sub lethally irradiated (350 cGy), and thereafter (48 hr post irradiation) transplanted with 50,000-100,000 hCD34+ cells, with or without additional ASCs (0.5×10⁶-1×10⁶) derived from placenta or adipose tissue (3-7 mice in each group), by intravenous injection to a lateral tail vein. Four to six weeks following transplantation the mice were sacrificed by dislocation and BM was collected by flushing both femurs and tibias with FACS buffer (50 ml PBS, 5 ml FBS, 0.5 ml sodium azid 5 percent). Human cells in the mice BM were detected by flow cytometry, and the percentage of the human and murine CD45 hematopoietic cell marker expressing cells in the treated NOD-SCID mice was effected by incubating cells with anti-human CD45-FITC (IQ Products, Groningen, The Netherlands). The lowest threshold for unequivocal human engraftment was designated at 0.5%. Detection of transplanted cells in mice treated with chemotherapy—6.5 week old male NOD-SCID mice (NOD.CB17/JhkiHsd-scid; Harlan, Rehovot Israel), maintained as described hereinabove for irradiated mice, were injected intraperitoneally with Busulfan (25 mg/kg—for 2 consecutive days). Two days following the second Busulfan injection, mice were injected with CD34⁺ cells alone, or together with 0.5×10⁶ ASCs, produced from the placenta. 3.5 weeks following transplantation, mice were sacrificed, and the presence of human hematopoietic cells was determined as described hereinabove for irradiated mice.

### Results

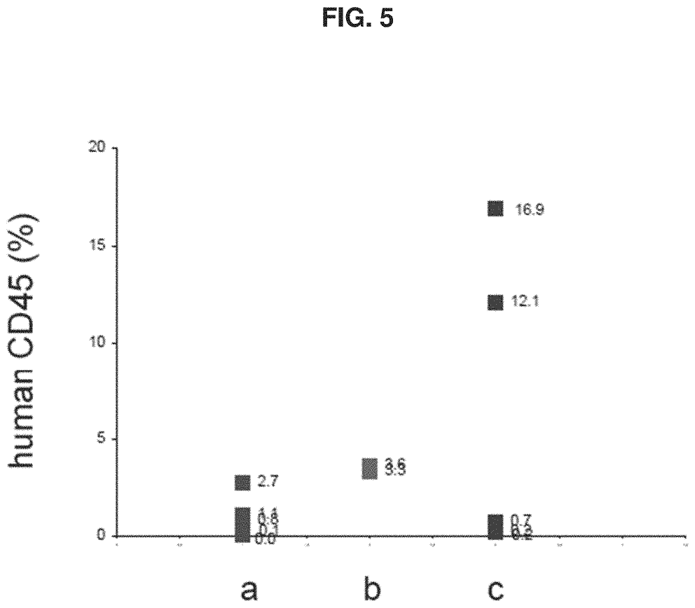

3D-ASC improved engraftment of HSC in irradiated mice—Human CD34+ hematopoietic cells and 3D-ASC derived from placenta or adipose were co-transplanted in irradiated NOD-SCID mice. Engraftment efficiency was evaluated 4 weeks following co-transplantation, and compared to mice transplanted with HSC alone. As is shown in Table 2 and FIG. 5, co-transplantation of 3D-ASC and UCB CD34+ cells resulted in considerably higher engraftment rates and higher levels of human cells in the BM of recipient mice compared to mice treated with UCB CD34+ cells alone.

TABLE 2

| Transplanted cells | Average h-CD45 | STDEV |
|---|---|---|
| CD34 | 3.8 | 7.9 |
| CD34 + 3D-ASC from placenta | 5.1 | 12.2 |
| CD34 + 3D-ASC from adipose | 8.7 | 9.6 |

3D-ASC improved engraftment of HSC in mice treated with chemotherapy—Human CD34+ hematopoietic cells were co-transplanted with 500,000 2D-ASC or 3D-ASC derived from placenta, into NOD-SCID mice pretreated with chemotherapy. Engraftment efficiency was evaluated 3.5 weeks following co-transplantation, and compared to mice transplanted with HSC alone. As is shown in Table 3, co-transplantation of ASC and UCB CD34+ cells resulted in higher engraftment levels in the BM of the recipient mice compared to UCB CD34+ cells alone. Moreover, as is shown in Table 3, the average level of engraftment was higher in mice co-transplanted with placenta derived adherent cells grown in the PluriX bioreactor system (3D-ASC) than in the mice co-transplantation with cells from the same donor, grown in the conventional static 2D culture conditions (flask).

TABLE 3

| Transplaned cells | Average h-CD45 | STDEV |
|---|---|---|
| CD34 | 0.9 | 1.1 |
| CD34 + conventional | 3.5 | 0.2 |
| 2D cultures from placenta | | |
| CD34 + 3D-ASC from placenta | 6.0 | 7.9 |

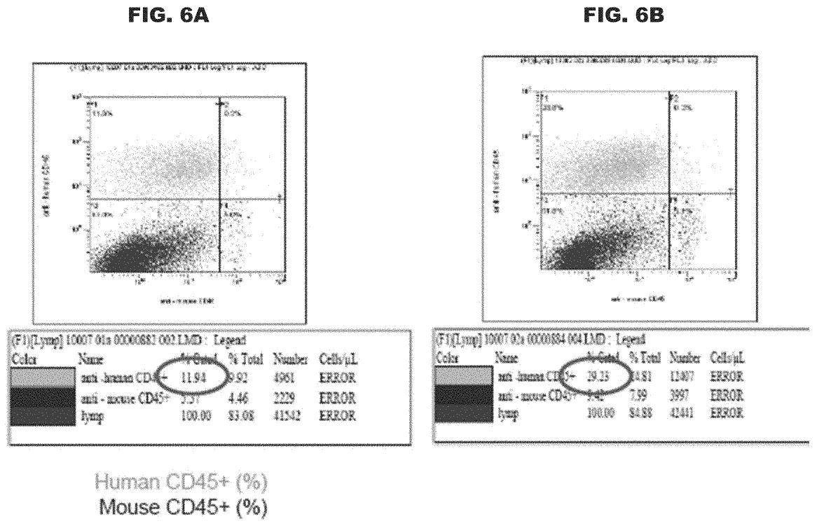

FACS analysis results shown in FIGS. 6A-B demonstrate the advantage of co-transplanting ASC with hHSCs (FIG. 6B), and the ability of ASC to improve the recovery of the hematopoietic system following HSC transplantation.

Taken together, these results show that ASCs may serve as supportive cells to improve hematopoietic recovery following HSCs transplantation (autologous or allogenic). The ability of the 3D-ASCs to enhance hematopoietic stem and/or progenitor cell engraftment following HSCs transplantation may result from the 3D-ASC ability to secrete HSC supporting cytokines that may improve the homing, self-renewal and proliferation ability of the transplanted cells, or from the ability of those cells to rebuild the damaged hematopoietic microenvironment needed for the homing and proliferation of the transplantable HSCs.

### Example 3

### The Suppression of Lymphocyte Response by 2D and 3D Cultured ASCs

Adherent stromal cells, and particularly 3D-ASCs, were found to suppress the immune reaction of human cord blood mononuclear cells in an MLR assay

### Materials and Experimental Procedures

Mixed lymphocyte reaction (MLR) assay—The immunosuppressive and immunoprivileged properties of 2D and 3D derived culturing procedures ASCs produced from the placenta, were effected by the MLR assay, which measures histocompatibility at the HLA locus, as effected by the proliferation rate of incompatible lymphocytes in mixed culturing of responsive (proliferating) and stimulating (unproliferative) cells. Human cord blood (CB) mononuclear cells (2×10⁵) were used as responsive cells and were stimulated by being co-cultured with equal amounts (10⁵) of irradiated (3000 Rad) human Peripheral Blood derived Monocytes (PBMC), or with 2D or 3D cultured adherent cells, produced from the placenta, or a combination of adherent cells and PBMCs. Each assay was replicated three times. Cells were co-cultured for 4 days in RPMI 1640 medium (containing 20% FBS under humidified 5% CO₂ atmosphere at 37° centigrade), in a 96-well plate. Plates were pulsed with 1 μC 3H-thymidine during the last 18 hr of culturing. Cells were then harvested over fiberglass filter and thymidine uptake was quantified with a scintillation counter.

### Results

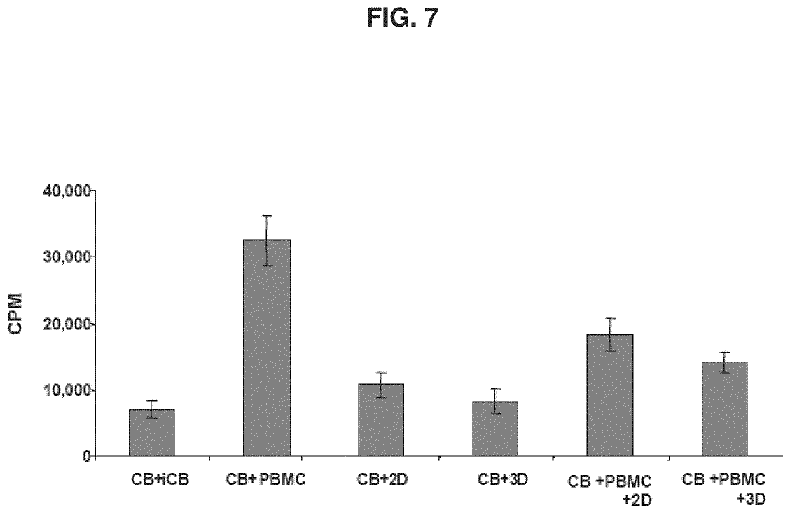

FIG. 7 shows the immune response of CB cells as represented by the elevated proliferation of these cells when stimulated with PBMCs, which, without being bound by theory, is probably associated with T cell proliferation in response to HLA incompatibility. However, a considerably lower level of immune response was exhibited by these cells when incubated with the described adherent cells. Moreover, the CB immune response to PBMCs was substantially reduced when co-incubated with these adherent cells. Thus, in a similar manner to MSCs, ASCs were found to have the potential ability to reduce T cell proliferation of donor cells, typical of GvHD. Although both cultures, 2D and 3D, reduced the immune response of the lymphocytes, and in line with the other advantages of 3D-ASCs described hereinabove, the 3D ASCs were more immunosuppressive.

### Example 4: Manufacture of 3D Adherent Cells

In order to provide large scale 3D adherent cells, a new manufacturing system was utilized referred to herein as Celligen.

### Materials and Experimental Methods

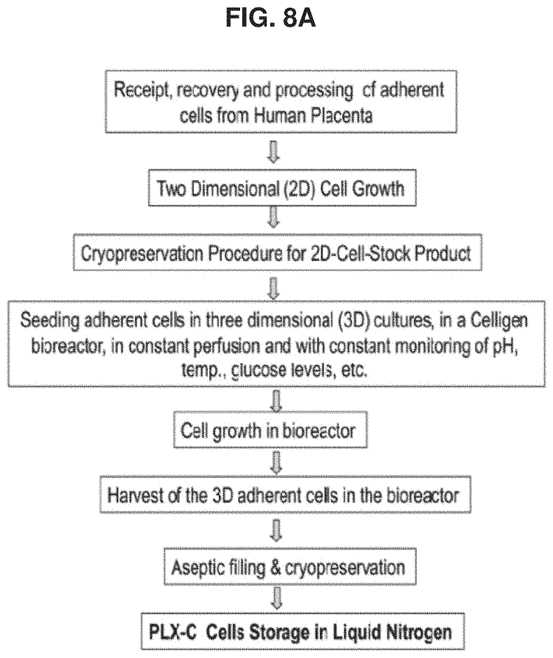

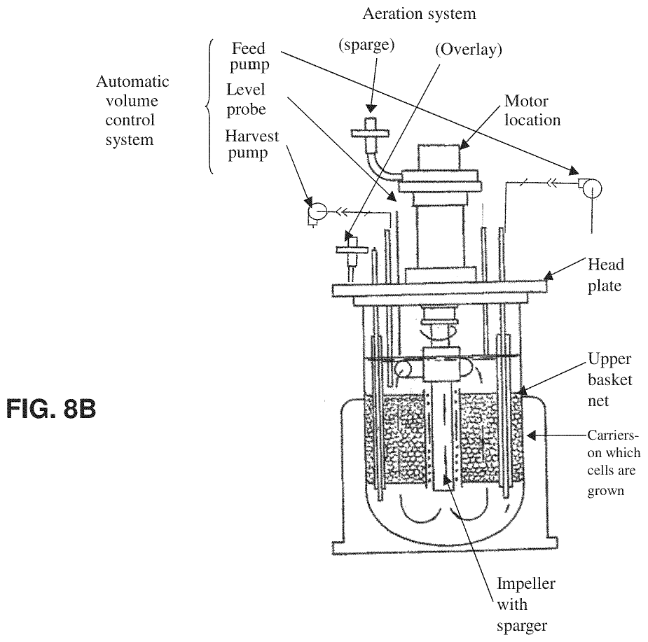

Celligen™ Plug Flow bioreactor—The production of adherent cells by Celligen™ (PLX-C cells) is composed of several major steps as illustrated in FIG. 8A. The process starts by collection of a placenta from a planned cesarean delivery at term.

Adherent cells are then isolated from whole placentas, grown in tissue culture flasks (2D cultures), harvested and stored in liquid nitrogen as 2D-Cell Stock (2DCS), the appropriate amount of 2DCS are thawed and seeded onto carriers in bioreactors for further expansion as 3D-culture. After 4-14 days of growth in the bioreactors, cells are harvested and cryopreserved in gas phase of liquid nitrogen as PLX-C.

All placentas obtained were received from the maternity ward under approval of the Helsinki Committee of the medical facility. Accordingly, all placenta donors signed an informed consent and Donor Screening and Donor Testing was performed.

Immediately after taking the placenta from the donor (during the caesarean procedure), it was placed in a sterile plastic bag and then in a Styrofoam box with ice packs. The placenta was delivered and immediately placed in a quarantine area until released to use by Quality Control (QC) and Quality Assurance (QA). All the following production steps were performed in a quarantine, clean room facility until QC approval of mycoplasma test results arrived and the cells were release for 2D cell growth.

To initiate the process, the whole placenta was cut into pieces under aseptic conditions under laminar flow hood, washed with Hank's buffer solution and incubated for 3 hours at 37° C. with 0.1% Collagenase (1 mg Collagenase/ml tissue). 2D cell medium (2D-Medium comprising DMEM supplemented with 10% FBS, fungizone 0.25 μg/ml and gentamycine 50 μm/ml) was added and the digested tissue was roughly filtered through a sterile metal strainer, collected in a sterile beaker and centrifuged (10 minutes, 1200 RPM, 4° C.). Using gentle pipeting, suspended cells were then washed with 2D-Medium supplemented with antibiotics, seeded in 80 cm2 flasks and incubated at 37° C. in a tissue culture incubator under humidified condition supplemented with 5% CO2. Following 2-3 days, in which the cells were allowed to adhere to the flask surface, they were washed with PBS and 2D-Medium was added.

Two Dimensional (2D) Cell Growth—Prior to the first passage, growth medium samples of 10% of the total flask number in quarantine was pooled and taken for mycoplasma testing. If cells were found to be negative for Mycoplasma (EZ-PCR Mycoplasma kit, Biological Industries, Israel), cells were released from quarantine. After 1-2 additional passages, cells were transferred to the 2D production clean room (2DP). Once in Room 2DP, culture was continued for another 3-5 passages. Sample was taken for immune phenotype after passage 4. Throughout the process, cultures were grown in 2D-Medium without antibiotics in a tissue culture incubator under humidified conditions with 5% CO2 at 37° C. After a total of 6-8 passages (9-16 cell doublings), cells were collected and cryopreserved as the 2D-Cell Stock (2DCS).

The first passage was usually carried out after 10-15 days. Beginning at passage 2 and continuing until passage 6-8, cells were passaged when the culture reached 70-80% confluence, usually after 3-5 days (1.5-2 doublings). The cells were detached from the flasks using 0.25% trypsin-EDTA (4 minutes at 37° C.) and seeded in a culture density of 3±0.2×10<3>cells/cm². The size of the tissue culture flasks raised as the passages proceed. The culturing process started in 80 cm² tissue culture flask, continued in 175 cm², then in 500 cm² (Triple flask) and finally the cells were seeded into Cell Factory 10 tray (6320 cm²).

Cryopreservation Procedure for 2D-Cell-Stock Product—For 2DCS cryopreservation, 2D-cultured cells were collected under aseptic conditions using 0.25% trypsin-EDTA. The cells were centrifuged (1200 RPM, 10′, 4° C.), counted and re-suspended in 2D-Medium.

For freezing, cell suspensions were diluted 1:1 with 2D-Freezing Mixture (final concentrations was 10% DMSO, 40% FBS and 50% 2D-Medium). Approximately 1.5-2.5×10⁹ cells were manufactured from one placenta. 4 ml of the cells were stored at a final concentration of 10×10⁶/ml in 5 ml cryopreservation polypropylene vials. The vials were labeled and transferred to a controlled rate freezer for a graduated temperature reducing process (1° C./min), after which they were transferred to storage in gas-phase of a liquid nitrogen freezer located in the Cold Storage Room. This material was referred to as the 2D-Cell Stock (2DCS) batch.

Initiation of the Three Dimensional (3D) Culture Procedures—To begin 3D culture, an appropriate amount (150±30×10⁶) of cells from 2DCS were thawed in the 2DP room and washed with 3D-Medium (DMEM with 10% FBS and 20 Mm Hepes) to remove DMSO prior to seeding in the prepared-in-advanced bioreactor systems. The content of each 2DCS vial was pipetted and diluted 1:9 with pre-warmed (37° C.) 3D-Medium. The cells were centrifuged (1200 RPM, 10′, 4° C.) and re-suspended again in 50-100 ml pre-warmed (37° C.) 3D-Medium in a 250 ml sterile bottle. A sample was taken and cells were counted using a Trypan Blue stain in order to determine cell number and viability. The cell suspension was transferred under a laminar flow hood into a 0.5 L seeding bottle. From the seeding bottle the cell suspension was transferred via sterile tubing to the bioreactor by gravitation.

Production of 3D-adherent cells in the Celligen Bioreactor (PLX-C)—3D growth phase was performed using an automatic CelliGen Plus® or BIOFLO 310 bioreactor system [(New Brunswick Scientific (NBS)] depicted in FIG. 8B. The bioreactor system was used for cultivation of cells, in which conditions were suitable for high cell concentrations. The cultivation process was carried out using a bioreactor in a perfusion mode. The lab scale bioreactor was constructed of two main systems—the control system and the bioreactor itself (vessel and accessories). The parameters of the process were monitored and controlled by a control console which included connectors for probes, motor and pumps, control loops for Dissolved Oxygen (DO), pH, perfusion and agitation (with a motor), a gases control system, water circulation and heating system for temperature control and an operator interface. The controlled process parameters (such as temperature, pH, DO etc.) could be displayed on the operator interface and monitored by a designated controller.

As noted above, 150±30×10⁶ cells from the cryopreserved 2DCS were thawed, washed and seeded in a sterile bioreactor. The bioreactor contained 30-50 gr carriers (FibraCel® disks, NBS), made of Polyester and Polypropylene and 1.5±0.1 L 3D-Medium. The growth medium in the bioreactor was kept at the following conditions: 37° C., 70% Dissolved Oxygen (DO) and pH 7.3. Filtered gases (Air, CO₂, N₂ and O₂) were supplied as determined by the control system in order to keep the DO value at 70% and the pH value at 7.3. For the first 24 hours, the medium was agitated at 50 Rounds Per Minutes (RPM) and increased up to 200 RPM by day 2. For the first 2-3 days, the cells were grown in a batch mode. Perfusion was initiated when the medium glucose concentration decreased below 550 mg/liter. The medium was pumped from the feeding container to the bioreactor using sterile silicone tubing. All tubing connections were performed under laminar flow using sterile connectors. The perfusion was adjusted on a daily basis in order to keep the glucose concentration constant at approximately 550±50 mg\liter. A sample of the growth medium was taken every 1-2 days for glucose, lactate, glutamine, glutamate and ammonium concentration determination (BioProfile 400 analyzer, Nova Biomedical). The glucose consumption rate and the lactate formation rate of the cell culture were used to measure cell growth rate. These parameters were then used to determine the harvest time based on accumulated experimental data.

The cell harvest process started at the end of the growth phase (4-10 days). Two samples of the growth medium were collected. One sample was prepared to be sent to an approved GLP laboratory for Mycoplasma testing according to USP and Eu standards, and the other one was transferred to a controlled rate freezer for a graduated temperature reducing process (1° C./min), after which they were transferred to storage in gas-phase of a liquid nitrogen freezer located in the Cold Storage Room, in case a repeat Mycoplasma testing was needed. These medium samples were considered as part of the Mycoplasma testing of the final product and the results were considered as part of the criteria for product release.

The 3D-grown culture was harvested as follows:

The bioreactor vessel was emptied using gravitation via tubing to a waste container. The vessel was opened, by removing the head plate, and the carriers were aseptically transferred from the basket to the upper basket net (see FIG. 8B). The bioreactor vessel was then closed and refilled with 1.5 L pre-warmed PBS (37° C.). The agitation speed was increased to 150 RPM for 2 minutes. The PBS was drained and the washing procedure was repeated twice.

In order to release the cells from the carriers, 1.5 L pre-warmed to 37° C. Trypsin-EDTA (Trypsin 0.25%, EDTA 1 mM) was added to the bioreactor vessel and carriers were agitated for 5 minutes in 150 RPM, 37° C. Cell suspension was collected to a 5 L sterile container containing 250 ml FBS. Cell suspension was divided to 4 500 ml sterile centrifuge tubes and a Mycoplasma test sample was withdrawn. Cells were aseptically filled and cryopreserved as PLX-C.

FACS analysis of membrane markers—cells were stained with monoclonal antibodies as previously described. In short, 400,000-600,000 cells were suspended in 0.1 ml flow cytometer buffer in a 5 ml test tube and incubated for 15 minutes at room temperature (RT), in the dark, with each of the following monoclonal antibodies (MAbs): FITC-conjugated anti-human CD29 MAb (eBioscience), PE-conjugated anti-human CD73 MAb (Becton Dickinson), PE-conjugated anti-human CD105 MAb (eBioscience), PE-conjugated anti-human CD90 MAb (Becton Dickinson), FITC-conjugated anti-human CD45 MAb (IQProducts), PE-conjugated anti-human CD19 MAb (IQProducts), PE-conjugated anti-human CD14 MAb (IQProducts), FITC conjugated anti-human HLA-DR MAb (IQProduct), PE-conjugated anti-human CD34 MAb (IQProducts), FITC-conjugated anti-human CD31 MAb (eBioscience), FITC-conjugated anti-human KDR MAb (R&D systems), anti-human fibroblasts marker (D7-FIB) MAb(ACRIS), FITC-conjugated anti-human CD80 MAb (BD), FITC-conjugated anti-human CD86 MAb (BD), FITC-conjugated anti-human CD40 MAb (BD), FITC-conjugated anti-human HLA-ABC MAb (BD), Isotype IgG1 FITC conjugated (IQ Products), Isotype IgG1 PE conjugated (IQ Products).

Cells were washed twice with flow cytometer buffer, resuspended in 500 μl flow cytometer buffer and analyzed by flow cytometry using FC-500 Flow Cytometer (Beckman Coulter). Negative controls were prepared with relevant isotype fluorescence molecules.

Mixed Lymphocyte Reaction (MLR)—2×10⁵ peripheral blood (PB) derived MNC (from donor A) were stimulated with equal amount of irradiated (3000 Rad) PB derived MNCs (from donor B). Increasing amounts of PLX-Cs were added to the cultures. Three replicates of each group were seeded in 96-well plates. Cells were cultured in RPMI 1640 medium containing 20% FBS. Plates were pulsed with 1 μC 3H-thymidine during the last 18 hrs of the 5-day culturing. Cells were harvested over a fiberglass filter and thymidine uptake was quantified with scintillation counter.

For CFSE staining, PB-MNC cells were stained for CFSE (Molecular Probes) for proliferation measurement before culturing. Cells were collected after 5 days and the intensity of CFSE staining was detected by Flow Cytometry.

### ELISA

ELISA was carried out as was previously described. In short, MNCs (isolated from peripheral blood) were stimulated with 5 μg/ml ConA (Sigma), 0.5 μg/ml LPS (SIGMA), or 10 μm/ml PHA (SIGMA) in the presence of PLX-C under humidified 5% CO2 atmosphere at 37° C. Supernatants were collected and subjected to cytokine analysis using ELISA kits for IFNγ (DIACLONE), TNFα (DIACLONE) and IL-10 (DIACLONE).

Expression of cellular markers on PLX-C cells—the surface antigens expressed by PLX-C were examined using monoclonal antibodies as described above. Results indicated that PLX-C cells were positive for the markers CD73, CD29 and CD105 and negative for the markers CD34, CD45, CD19, CD14 and HLA-DR. The immune phenotype test specifications were set as: ≥90% for all positive markers and ≤3% for all negative markers.

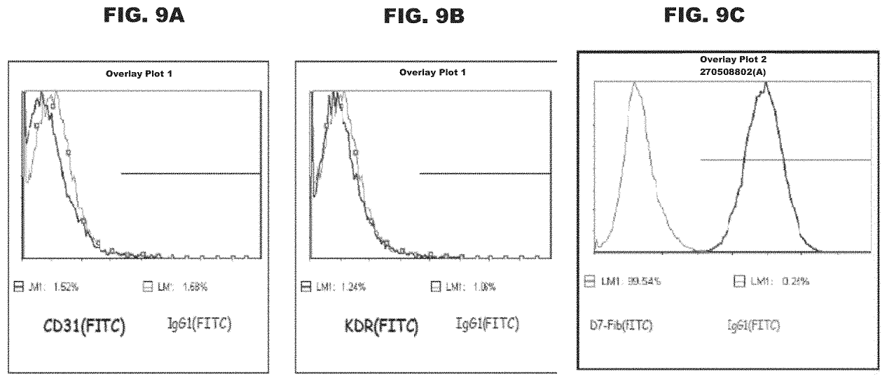

Furthermore, as shown in FIGS. 9A-B, PLX-C cultures did not express endothelial markers as shown by negative staining for the two endothelial markers CD31 and KDR. However, PLX-C expression of a fibroblast-typical marker was evident (expression of D7-fib, FIG. 9C).

Immunogenecity and immunomodulatory properties of PLX-C cells—As PLX-C is comprised of adherent cells derived from placenta, it is expected to express HLA type I, which is expressed by all cells of the body and is known to induce an alloreactive immune response. HLA type II and other co-stimulatory molecules are typically expressed only on the surface of Antigen Presenting Cells (APCs).

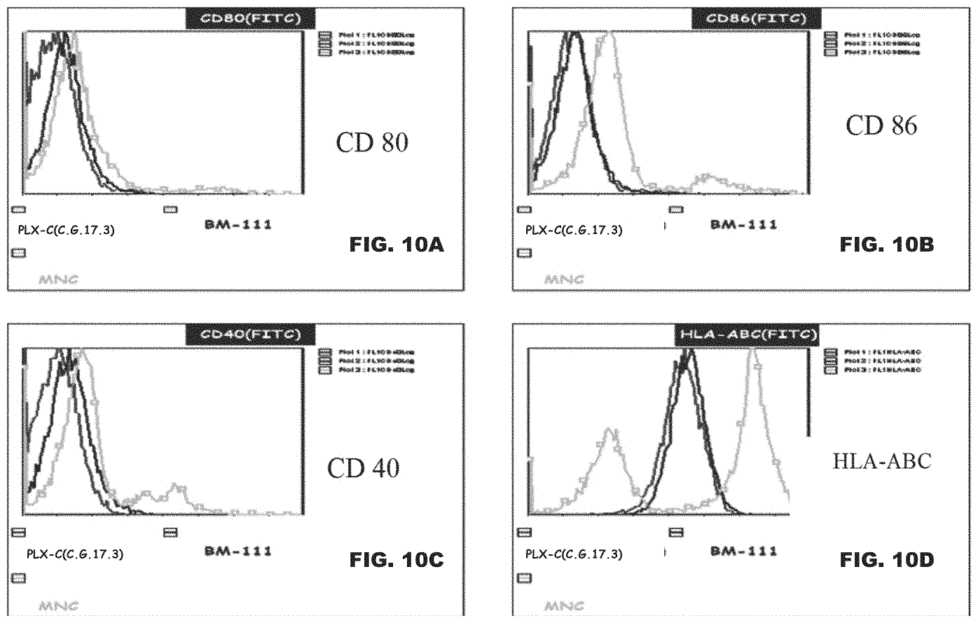

To examine the immunogenicity of the PLX-C cells, analysis of the expression of co-stimulatory molecules on the surface of these cells was performed. FACS analysis demonstrated the absence of detectable CD80, CD86 and CD40 on the PLX-C cell membranes (FIGS. 10A-C). Moreover, PLX-C expressed low levels HLA class I as detected by staining for HLA A/B/C (FIG. 10D). The PLX-C were similar to bone marrow (BM) derived MSCs in their lack of expression of stimulatory and co-stimulatory molecules (as shown in FIGS. 10A-D).

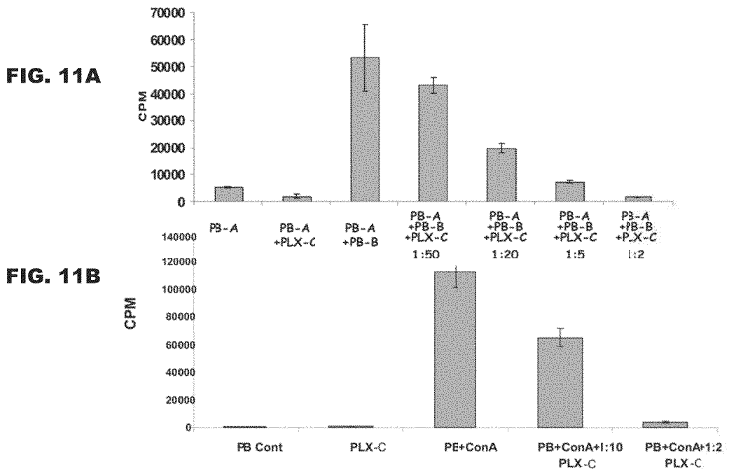

To further investigate the immunogenecity as well as the immunomodulation properties of PLX-C cells, Mixed Lymphocyte Reaction (MLR) tests were performed. As shown in FIG. 11A-B, PLX-C cells both escape allorecognition, and reduce T cell response, as measured by thymidine incorporation. Furthermore, the reduction in lymphocytes proliferation (evaluated by CPM measurement) was higher as the number of PLX-C cells increased (in a dose dependent manner). PLX-C also reduced lymphocyte proliferation following mitogenic stimuli, such as Concavalin A (Con A, FIG. 11B) and Phytohemagglutinin (PHA), and non-specific stimulation by anti-CD3, anti-CD28.

In order to investigate the mechanism of action by which PLX-C immunomodulate lymphocyte proliferation, and to see if this action is mediated via cell to cell interaction or cytokines secretion, PB derived Mononuclear cells (MNCs) were stimulated by PHA using the transwell method (which prevents cell to cell contact but enables the diffusion of cytokines between the two compartments). Results showed that the inhibition of proliferation maintained even when cell to cell contact was inhibited.

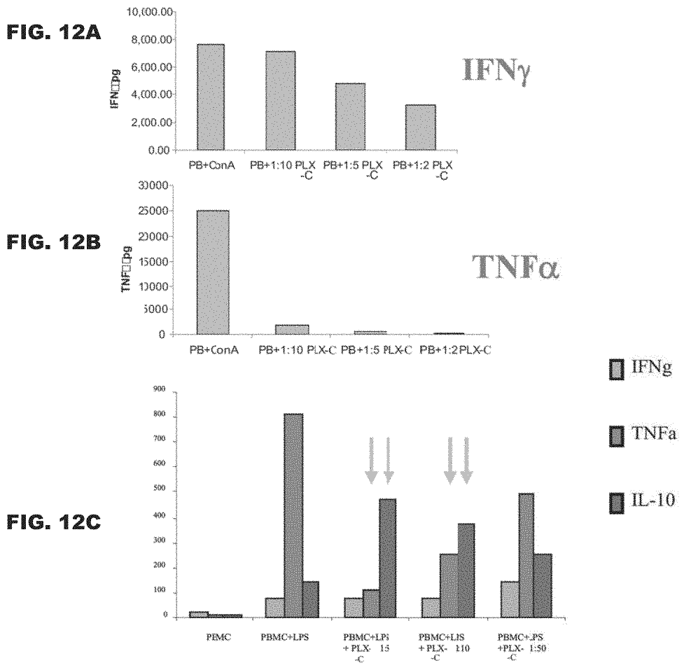

Cytokines secretion—as depicted hereinabove, PLX-C reduce the proliferation rate of lymphocytes, probably through soluble factors. Further investigation of the cytokines secreted by lymphocytes in response to PLX-C was performed to elucidate the mechanism of action of PLX-C. As depicted in FIGS. 12A-B, culturing of mononuclear cells with PLX-C slightly reduces the secretion of the pro-inflammatory cytokine INFγ and dramatically reduces the secretion of TNFα (even in the presence of low amounts of PLX-C). In addition, following lipopolysaccharide (LPS) stimulation, PB derived MNCs secretion of IL-10 increased in the presence of PLX-C, while the secretion level of TNFα decreased, in a dose dependent manner (FIG. 12C).

### Example 5: No Significant T Cell Alloreactivity in Patients Treated with PLX Cells

In general, engraftment of cells that are unmatched in their histocompatibility antigens (HLA) to a recipient (i.e., allogeneic cells) will generate a robust host versus graft response leading to a rapid elimination of the cells from the recipient's body. The major immune cell driving this rejection response is the T cell. T cells can specifically identify foreign HLA bearing cells and destroy them.

ASCs derived from placenta have immunosuppressive characteristics and have been shown to exert therapeutic properties in various pre-clinical animal disease models despite their xenogeneic origin (i.e., engraftment between individuals of different species). ASCs prepared in accordance with Example 4 are thus being evaluated in a dose-escalating phase I clinical trial as an allogeneic product.

Specifically, two phase I studies using ASCs, intended for treatment of critical limb ischemia (CLI), were designed to evaluate safety, including immunological profile associated with local administration. These open-label, dose-escalation studies were performed in parallel in the EU and U.S. The design of the studies is similar, but not identical. For example, the follow up period and dose escalation schedules differ following regulatory requirements and previous experience of the clinical sites. The clinical follow-up period for both studies is three months after treatment; however, in the EU, the patients are observed for 24 months, versus 12 months in the U.S.

Altogether five dosing groups were evaluated. For the low dose group in the U.S., ASCs were multiply injected during one course. For the high dose group the additional dosing was achieved by administering the cells by multiple injections during two courses, two weeks apart. In contrast, in the EU, the higher dose was administered in a single course of multiple injections, using higher volumes of cells per injection.

In the U.S., ASCs were administered via 30 intramuscular (IM) injections delivered to the affected leg for the low dose treatment group, while for the higher dose, ASCs was administered twice (two courses, two weeks apart), with 30 IM injections delivered to affected leg in each course. In the EU study, all three treatment groups were treated with 30 IM injections delivered to the affected leg.

In order to evaluate whether patients treated with ASCs had developed a specific T cell response to the ASCs, a series of blood tests was performed before and following cell injections. Peripheral blood mononuclear cells (PBMCs) from treated patients were subjected to an Enzyme Linked Immuno-Sorbent SPOT (ELISPOT) Assay in order to evaluate the frequency of their anti-ASC T cell responses.

The ELISPOT assay is based on detecting interferon gamma secreting cells. Peripheral blood mononuclear cells (PBMCs) are separated from whole blood and incubated with the tested T cell antigen (e.g., peptide, protein or whole cells). The cells are then plated on a membrane that was pre-coated with interferon-specific antibodies. Specific T cells that respond to the tested component will then become stimulated and secrete interferon gamma protein. The anti-interferon antibodies on the membrane will then capture interferon in proximity to the secreting cell. Next, the cells are washed away and the membranes are incubated with an enzyme conjugated anti interferon gamma antibody. After washing unbound antibodies, a substrate is added and transformed by the enzyme into a black precipitant on the membrane. Thus, each spot on the membrane represents an interferon gamma secreting cell. The number of spots per total PBMCs plated represents the frequency of T cells that are specific for a given antigen.

This assay was performed using a commercially available interferon ELISPOT kit (EliSpot Basiskit ELSP 5500, AID, Strassberg, GmbH) according to the manufacturer's protocol. Briefly, whole blood was drawn from patients at the day of injection (V2), and 24 hours (V3), and one week (V4) after ASC administration. PBMCs were separated from whole blood by Ficoll gradient centrifugation and stored at −80° C. until tested. 300,000 cells were then stimulated with CMV IE-1, CMV pp65, EBV peptides and allogeneic cells (as positive control) and PLX cells or left un-stimulated as a negative control. Following stimulation, cells were incubated in interferon gamma ELISPOT well plates. Cells were then washed away and a biotinilated anti interferon gamma antibody was added to plates. After washing away residual antibody, a streptavidin-conjugated alkaline phosphatase was added. The reagent was washed and the alkaline phosphatase substrate NBT/BCIP was then added to form a blue-black precipitant. Spots were counted and analyzed.

The following Table 4 shows the HLA genotypes of two batches (P110209 and P040509) of ASCs used in these experiments. As shown in the table, P110209 is a homogenous population as reflected in only two alleles at each locus. In contrast, P040509 is a mixed population having three alleles at the HLA-A and HLA-B loci. In this case the cells are derived from both maternal and fetal portions of the placenta.

TABLE 4

| Batch | Class I | | | | | | Class II | | | |
|---|---|---|---|---|---|---|---|---|---|---|
| no. | A | | | B | | | DR | | DQ | |
| P110209 | A*02 | A*11 | | B*35 | B*52 | | DRB1*0301 | DRB1*15 | DQB1*02 | DQB1*06 |
| P040509 | A*11 | A*24 | A*31 | B*35 | B*40 | B*51 | DRB1*11 | DRB1*15 | DQB1*03 | DQB1*06 |

In this assay, 300,000 PBMCs from PLX treated patients were stimulated in vitro with CMV and EBV peptides, PLX cells, allogeneic cells or left unstimulated. Cells were subjected to interferon gamma ELISPOT assay and number of spots per treatment were counted. The results are shown in the Table 5.

TABLE 5

| | | | Antigen-specific T cell response | | | | | |
|---|---|---|---|---|---|---|---|---|
| Patient | Batch | Visit | CMV | CMV | | PLX- | Back- | PLX- |
| symbol | number | number | IE-1 | pp65 | EBV | reactive | ground | Background |
| hau 990 | P110209 R89 | V2 | 1 | 0 | 14 | 0 | 3 | −3 |
| | | V3 | 1 | 1 | 11 | 0 | 0 | 0 |
| | | V4 | 1 | 1 | 11 | 0 | 2 | −2 |
| zaq 372 | P110209 R89 | V2 | 17 | TNTC* | 42 | 4 | 0 | 4 |
| | | V3 | 83 | TNTC | 193 | 7 | 7 | 0 |
| | | V4 | 43 | TNTC | 120 | 6 | 5 | 1 |
| eqo 406 | P040509 R12 | V2 | 13 | 164 | 7 | 1 | 4 | −3 |
| | | V3 | 10 | 136 | 7 | 6 | 3 | 3 |
| | | V4 | 0 | 3 | 0 | 1 | 0 | 1 |
| ooc 517 | P040509 R34 | V2 | 2 | 45 | 0 | 0 | 0 | 0 |
| | | V3 | 1 | 0 | 0 | 0 | 0 | 1 |
| | | V4 | 0 | 0 | 0 | 0 | 0 | 0 |

The results in Table 5 show that most patients reacted to CMV or EBV peptides as expected because most of the adult population has been exposed to those viruses and the existence of anti CMV or EBV T cells is prevalent. In addition, the number of spots does not increase after treatment, indicating that immunomodulation characteristics of PLX cells have no systemic effect on anti CMV and EBV memory T cell response. Throughout the treatment the no or very low T cell reactivity to PLX cells is observed in either the homogenous or mixed cell populations. This result demonstrates for the first time that mixed populations of ASCs derived from human placenta can be administered to humans without eliciting an immune response. Thus, this result demonstrates that mixed populations of ASCs can be created from one or more placentas and administered together to human subjects for therapeutic indications.

### Example 6: Mixed Populations of PLX-C Cells Also Inhibit Mitogen-Induced T Cell Proliferation

The phytohemagglutinin (PHA) test measures the rate by which placenta derived cells reduce the proliferation of lymphocytes following mitogenic PHA stimuli. The objective of this study was to evaluate the immunosuppressive properties of placenta derived cells from a single donor in comparison to a cell population derived from multiple donors according to their ability to reduce the proliferation of PHA-stimulated lymphocytes. The mixed HLA populations were either co-cultured in vitro prior to the PHA test or grown separately and mixed just prior PHA testing.

### Materials and Methods

a. Thaw the PLX vials (see Table 6) in RPMI medium.
b. After discarding the supernatant, suspend the pellet of each tube, add 3 ml of RPMI medium to each tube and transport 0.3 ml sample from each tube to a cedex cup (300 μl for CEDEX count).
c. Count the cells (the minimal required cells amount is 1.5*10⁶).
d. Transport to Eppendorf tubes 0.4*10⁶ cells from each batch and fill up with RPMI to final volume of 1 ml.
e. Seed each batch at the 40,000 cell/well concentration, 100 μl/well in 96 wells plate in triplicates.
f. Perform ×2 dilution of the 40,000 cell/well concentration by adding 0.7 ml RPMI medium to the Eppendorf tubes and seed each batch at the 20,000 cell/well concentration, 100 μl/well in 96 wells plate in triplicates.
g. Thaw frozen PBMCs in RPMI medium.
h. Dislodge the acquired pellet and take a sample for direct count, diluted 1:10 in Turk's Buffer.
i. Count by hemacytometer.
j. Adjust PBMCs concentration to 2*10⁶ cells/ml using RPMI medium.
k. Fill the first 2 groups of wells (=6 wells) of 96 wells plate with 100 μl RPMI medium without cells (for PBMCs control groups—group A (negative) and group B (positive)).
l. Add 100 μl of the PBMCs suspension to the first group of wells (200,000 PBMCs/well—group A, negative control).
m. Remove the required amount of PBMCs to a new 50 ml tube and add 20 μl PHA stock solution (1 mg PHA/ml in PBS) per each 1 ml of PBMCs suspension.
n. Culture PHA stimulated PBMCs in 96-well plate (except the first triplicate), 100 μl suspension/well.
o. Incubate the plate for 3 days in an incubator (37° C., 5% CO₂).
p. To quantify PBMCs proliferation use Click-iT EdU Cell Proliferation Assay (Invitrogen Cat. no. C35002)

### Results

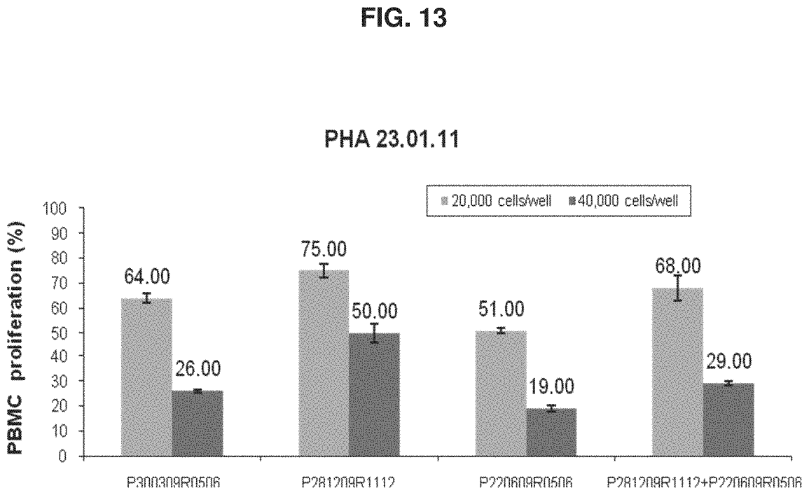

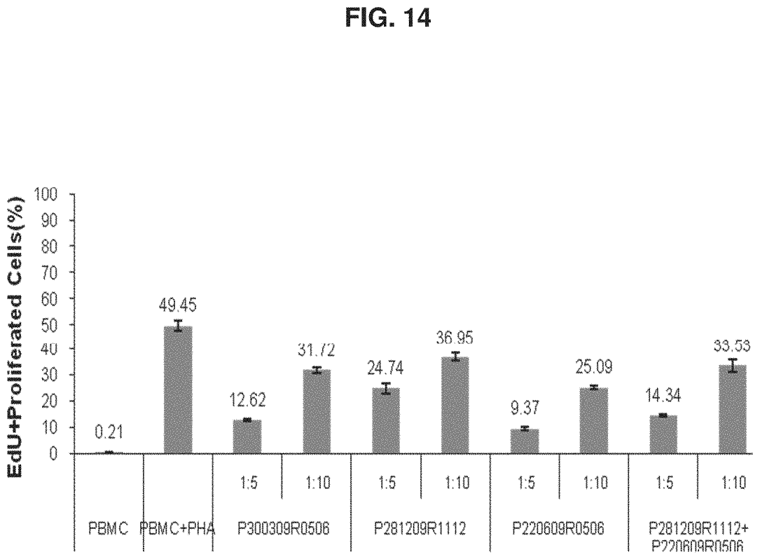

Culturing PBMC in the presence of PHA results in proliferation of those cells. FIG. 13 shows the PHA-induced proliferation rate of PBMC cultured in the presence of different numbers of PLX cells from three different donors (P300309R0506; P281209R1112; and P220609R0506) or a mixture of two donors (P281209R1112+P220609R0506). FIG. 14 presents the average PBMC proliferation for PBMC cultured without addition of PHA, PBMC cultured with PHA but without addition of any PLX cells, and PBMC cultured in the presence of PHA and PLX cells from different donors. The percentage of proliferation using the proliferation of the PBMC+PHA as 100% is shown in Table 6.

TABLE 6
PBMC Proliferation (%) in presence of different batches of PLX.

| | | PBMC Proliferation (%) | | | |
|---|---|---|---|---|---|
| | | 20,000 cell/well | | 400,000 cell/well | |
| Group | Batch | AVG | SD | AVG | SD |
| C | P300302R0506 | 64 | 1.94 | 26 | 0.63 |
| D | P281209R1112 | 75 | 2.77 | 50 | 3.69 |
| E | P220609R0506 | 51 | 1.13 | 19 | 1.25 |
| F | P281209R1112 + | 68 | 4.9 | 29 | 0.76 |
| | P220609R0506 | | | | |

Mixing PLX cells from two donors resulted in an inhibition of PHA-stimulated proliferation that was intermediate to the effect observed when PLX cells for either donor were tested individually. Thus, PLX cells from different donors can be mixed and the PHA-induced inhibitory effect is still maintained.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method of producing a pharmaceutical composition, comprising:
a) generating a population of adherent stromal cells by a method comprising the steps of:
i. culturing adherent stromal cells from placenta or adipose tissue under three-dimensional culturing conditions, which support cell expansion, wherein said three-dimensional culturing conditions comprise: (a) a 3D bioreactor; and (b) an adherent material selected from the group consisting of a polyester, a polyalkylene, a polyfluorochloroethylene, a polyvinyl chloride, and a polysulfone; and
ii. obtaining the adherent stromal cells from the three-dimensional culturing conditions; and
b) adding a cryoprotectant and at least one pharmaceutically acceptable excipient to said population of adherent stromal cells.

2. The method of claim 1, wherein said three-dimensional culturing conditions are effected under a continuous flow of a culture medium.

3. The method of claim 1, wherein said adherent material is a non-cytotoxic material having a chemical structure which may retain the cells on a surface that has a shape selected from the group consisting of squares, rings, discs, and cruciforms.

4. The method of claim 1, wherein said adherent material when used in a plug-flow bioreactor is in the form of non-woven fiber sheets having a thickness of about 50-1000 micron, or sheets of open-pore foamed polymers.

5. The method of claim 1, wherein said adherent material is in the form of a bed of randomly packed substrates, each substrate comprising a fibrous matrix bonded to a porous support sheet, each said matrix comprising a physiologically acceptable three-dimensional network of fibers in the form of a sheet having a pore volume as a percentage of total volume of from 40-95% and a pore size of from 10 microns to 100 microns, the overall height of the matrix being from 50 microns to 500 microns.

6. The method of claim 1, wherein said adherent stromal cells are allowed to adhere to an adherent material, to thereby isolate adherent cells, prior to said culturing in 3D culturing conditions.

7. The method of claim 1, wherein said adherent material has an adherent surface with a shape selected from the group consisting of squares, rings, discs, and cruciforms.

8. The method of claim 1, wherein said adherent material is polyester.

9. The method of claim 1, wherein said adherent stromal cells are viable.

10. The method of claim 1, wherein the adherent stromal cells are derived from placenta.

11. The method of claim 1, wherein the adherent stromal cells are derived from adipose tissue.

12. The method of claim 1, wherein the adherent stromal cells from placenta or adipose tissue that have been cultured under three-dimensional culturing conditions secrete a higher level of at least one cytokine selected from the group consisting of Flt-3 ligand, IL-6, and stem cell factor (SCF) than that secreted by adherent stromal cells from placenta or adipose tissue that have been cultured under two-dimensional culturing conditions.

13. A method of expanding cells, comprising:
(i) culturing adherent stromal cells from placenta or adipose tissue under three-dimensional culturing conditions, which support cell expansion, wherein said three-dimensional culturing conditions comprise: (a) a 3D bioreactor; and (b) an adherent material selected from the group consisting of a polyester, a polyalkylene, a polyfluorochloroethylene, a polyvinyl chloride, and a polysulfone; and
(ii) obtaining the adherent stromal cells from the three-dimensional culturing conditions.

14. The method of claim 13, wherein said three-dimensional culturing conditions are effected under a continuous flow of a culture medium.

15. The method of claim 13, wherein said adherent material when used in a plug-flow bioreactor is in the form of non-woven fiber sheets having a thickness of about 50-1000 micron, or sheets of open-pore foamed polymers.

16. The method of claim 13, wherein said adherent material is in the form of a bed of randomly packed substrates, each substrate comprising a fibrous matrix bonded to a porous support sheet, each said matrix comprising a physiologically acceptable three-dimensional network of fibers in the form of a sheet having a pore volume as a percentage of total volume of from 40-95% and a pore size of from 10 microns to 100 microns, the overall height of the matrix being from 50 microns to 500 microns.

17. The method of claim 13, wherein said adherent stromal cells are allowed to adhere to an adherent material, to thereby isolate adherent cells, prior to said culturing in 3D culturing conditions.

18. The method of claim 13, wherein said adherent stromal cells are viable.

19. The method of claim 13, wherein the adherent stromal cells are derived from placenta.

20. The method of claim 13, wherein the adherent stromal cells are derived from adipose tissue.
